(19) United States
(12) Patent Application Publication
(10) Pub. No.: US 2024/0165217 A1
(43) Pub. Date: May 23, 2024

(54) METHOD OF ELICITING AN IMMUNE RESPONSE AGAINST RESPIRATORY SYNCYTIAL VIRUS

(71) Applicant: CODAGENIX INC., Farmingdale, NY (US)

(72) Inventors: Steffen Mueller, Great Neck, NY (US); John Robert Coleman, Blauvelt, NY (US); Sybil Tasker, Jackson, NH (US); Anna Kushnir, Washington, DC (US)

(73) Assignee: CODAGENIX INC., Farmingdale, NY (US)

(21) Appl. No.: 18/283,130

(22) PCT Filed: Apr. 8, 2022
(86) PCT No.: PCT/US2022/024068

§ 371 Date: Sep. 20, 2023

Related U.S. Application Data
(60) Provisional application No. 63/172,540, filed on Apr. 8, 2021.

(51) Int. Cl. A61K 39/12; A61K 39/00; A61P 37/02

(52) U.S. Cl. CPC A61K 39/12; A61P 37/02; A61K 2039/5254; A61K 2039/543; A61K 2039/545; A61K 2039/575; C12N 2760/18534; C12N 2760/18562; C12N 2760/18571

(57) ABSTRACT

The present invention provides for methods of vaccination and eliciting a IgA immune response and cellular immune response, including T-cell immune response by administering a deoptimized respiratory syncytial virus (RSV).

### CROSS-REFERENCE TO RELATED APPLICATIONS

This application includes a claim of priority under 35 U.S.C. § 119(e) to U.S. provisional patent application No. 63/172,540, filed Apr. 8, 2021, the entirety of which is hereby incorporated by reference.

### FIELD OF INVENTION

This invention relates to vaccines and providing protective immune responses against respiratory syncytial virus (RSV).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Respiratory syncytial virus is a non-segmented, negative-sense single-stranded enveloped ribonucleic acid (RNA) virus that belongs to the family of Pneumoviridae (formerly Paramyxoviridae). It is spread via respiratory secretions through close contact with infected people or contact with contaminated surfaces or objects. The humoral immune response normally results in the development of anti-RSV neutralizing antibody titers, but these are often suboptimal during an infant's initial infection. The cellular immune response to RSV infection is also important for the clearance of virus. This immune response, vital for host defense against RSV, is also implicated in the immunopathogenesis of severe lower respiratory tract RSV bronchiolitis. The incubation period is 3 to 5 days and viral shedding usually occurs for 3 to 8 days. It is spread via respiratory secretions through close contact with infected people or contact with contaminated surfaces or objects. Infection can occur when infectious particles contacts mucous membranes of the eyes, mouth, or nose, and possibly through the inhalation of droplets generated by a sneeze or cough.

While RSV has long been known to be a major pathogen in young children, there is growing recognition of the threat of RSV in older adults. A decline in immune function with aging increases the risk of different infectious diseases, including RSV and older adults living in the general community are susceptible to respiratory illness from RSV with subsequent increased risk of emergency department visits or hospitalization. In a retrospective epidemiological review of 10 studies reporting incidence proportions of laboratory-confirmed RSV in medically-attended older adults, RSV was estimated to be the causative agent in up to 12% of acute respiratory illness in older adults unselected for comorbidities, with variations in clinical setting and by year. Medically-attended-RSV incidence among older adults not selected for having underlying health conditions was observed to increase with increasing age and in hospitalized adults with chronic cardiopulmonary diseases, 8% to 13% were infected with RSV during winter seasons. Overall, the duration of hospitalizations for RSV in older adults was 3 to 6 days, with substantial proportions of patients requiring intensive care unit admission and mechanical ventilation. Among older adults hospitalized with RSV, the mortality rate was 6% to 8%.

Currently, there are no approved RSV vaccines, and there remains a need in the art to provide methods of vaccinating individuals, especially children and older adults.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of eliciting an immune response in a subject, comprising intranasally administering one or more doses of an effective amount of a composition comprising a deoptimized respiratory syncytial virus (RSV) to the subject to elicit the immune response, wherein the effective amount is about 10³-10⁹ focus forming units (FFU) of the deoptimized RSV, wherein the immune response comprises IgA immune response, cellular immune response or both.

In various embodiments, intranasally administering one or more doses of the effective amount of the composition can comprise administering two doses, wherein a second dose is administered about 28 days after a first dose. In various embodiments, intranasally administering one or more doses of the effective amount of the composition can comprise administering two doses, wherein a second dose is administered at least 28 days after a first dose. In various embodiments, intranasally administering one or more doses of the effective amount of the composition can comprise administering two doses, wherein a second dose is administered about 28-35 days after a first dose.

In various embodiments, intranasally administering can comprise administering via nose drops. In various embodiments, intranasally administering can comprise administering via nasal spray.

In various embodiments, the effective amount can be about 10⁵. In various embodiments, the effective amount can be about 2×10⁵. In various embodiments, the effective amount can be about 10⁶. In various embodiments, the effective amount can be about 2×10⁶.

In various embodiments, one dose of the effective amount can be provided in a volume of about 1 mL. In various embodiments, one dose of the effective amount can be administered in about four aliquots.

In various embodiments, a first aliquot can be administered in a first nostril, and a second aliquot can be administered in a second nostril within 10 minutes of the first aliquot being administered in the first nostril, and wherein a third aliquot and a fourth aliquot can be administered in to the first nostril and the second nostril, in either order, about 5-20 minutes after the second aliquot is administered into the second nostril.

In various embodiments, a first aliquot can be administered in a first nostril, and a second aliquot can be administered in a second nostril within 5 minutes of the first aliquot being administered in the first nostril, and wherein a third aliquot and a fourth aliquot can be administered in to the first nostril and the second nostril, in either order, about 10-15 minutes after the second aliquot is administered into the second nostril.

In various embodiments, the cellular immune response can comprise a development of a T-cell response. In various embodiments, the development of the T-cell response can be in comparison to a reference level of response based on a placebo group or in comparison to a prevaccination timepoint for the subject.

In various embodiments, an enzyme-linked immune absorbent spot (ELISpot) analysis of peripheral blood mononuclear cells (PBMCs) from the subject can show at least a 1.3 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject can show at least a 1.4 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject can show at least a 1.5 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject can show at least a 1.6 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject can show at least a 1.7 fold increase of spot forming units (SFU) between a baseline value and a highest value during a post vaccination timepoint.

In various embodiments, the effective amount can be about 10⁶ FFU and eliciting the immune response can comprise a statistically significant increase in T-cell response as compared to a prevaccination timepoint. In various embodiments, the effective amount can be about 2×10⁶ FFU and eliciting the immune response can comprise a statistically significant increase in T-cell response as compared to a prevaccination timepoint.

In various embodiments, the effective amount can be about 10⁵ FFU and eliciting the immune response can comprise an increase in T-cell response as compared to a prevaccination timepoint. In various embodiments, the effective amount can be about 2×10⁵ FFU and eliciting the immune response can comprise an increase in T-cell response as compared to a prevaccination timepoint.

In various embodiments, the effective amount can be about 10⁶ FFU and eliciting the immune response can comprise at least a 2.4 fold rise in IgA levels in the subject. In various embodiments, the effective amount can be about 2×10⁶ FFU and eliciting the immune response can comprise at least a 2.4 fold rise in IgA levels in the subject.

In various embodiments, the effective amount can be about 10⁵ FFU and eliciting the immune response can comprise at least a 1.1 fold rise in IgA levels in the subject. In various embodiments, the effective amount can be about 2×10⁵ FFU and eliciting the immune response can comprise at least a 1.1 fold rise in IgA levels in the subject.

In various embodiments, the effective amount can be about 10⁵-10⁶ FFU and eliciting the immune response can comprise at least a 1.3 fold rise in IgA levels in the subject. In various embodiments, the effective amount can be about 2×10⁵ to 2×10⁶ FFU and eliciting the immune response can comprise at least a 1.3 fold rise in IgA levels in the subject.

In various embodiments, the deoptimized RSV can comprise the genome or anti-genome having the sequence of SEQ ID NO: 1.

In various embodiments, the subject can be under the age of 18. In various embodiments, the subject can be under the age of 13. In various embodiments, the subject can be under the age of 7. In various embodiments, the subject can be under the age of 2. In various embodiments, the subject can be between 18-49 years old. In various embodiments, the subject can be at least 50 years old.

Various embodiments provide for a method of eliciting an T-cell immune response in a subject, comprising: intranasally administering a first dose of an effective amount of a composition comprising a deoptimized respiratory syncytial virus (RSV) to the subject to elicit the T-cell immune response; and intranasally administering a second dose of the effective amount of the composition comprising the deoptimized RSV about 28-35 days after the first dose, wherein the effective amount is about 10⁶ or about 2×10⁶ focus forming units (FFU) of the deoptimized RSV, wherein the deoptimized RSV comprises the genome or anti-genome having the sequence of SEQ ID NO: 1, and wherein eliciting the T-cell immune response comprises an increase in T-cell response as compared to a prevaccination timepoint.

Various embodiments provide for a method of eliciting an T-cell immune response in a subject, comprising: intranasally administering a first dose of an effective amount of a composition comprising a deoptimized respiratory syncytial virus (RSV) to the subject to elicit the immune response; intranasally administering a second dose of the effective amount of the composition comprising the deoptimized RSV about 28-35 days after the first dose, wherein the effective amount is about 10⁵ or about 2×10⁵ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint.

Various embodiments provide for a method of eliciting an IgA immune response, cellular immune response or both in a subject, comprising intranasally administering a first dose of an effective amount of a composition comprising a deoptimized respiratory syncytial virus (RSV) to the subject to elicit the immune response; and intranasally administering a second dose of the effective amount of the composition comprising the deoptimized RSV about 28-35 days after the first dose; wherein the effective amount is about 2×10⁵ focus forming units (FFU) of the deoptimized RSV, wherein the deoptimized RSV comprises the genome or anti-genome having the sequence of SEQ ID NO:1, and wherein an enzyme-linked immune absorbent spot (ELISpot) analysis of peripheral blood mononuclear cells (PBMCs) from the subject shows at least a 1.3 fold increase of spot forming units (SFU) between a baseline value and a highest value during a post vaccination timepoint.

Various embodiments provide for a method of eliciting an IgA immune response, cellular immune response or both in a subject, comprising intranasally administering a first dose of an effective amount of a composition comprising a deoptimized respiratory syncytial virus (RSV) to the subject to elicit the immune response; and intranasally administering a second dose of the effective amount of the composition comprising the deoptimized RSV about 28-35 days after the first dose; wherein the effective amount is about 2×10⁶ focus forming units (FFU) of the deoptimized RSV, wherein the deoptimized RSV comprises the genome or anti-genome having the sequence of SEQ ID NO:1, and wherein eliciting the immune response comprises at least a 2.4 fold rise in IgA levels in the subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1B show Total Spot counts of ELISPOT for the Placebo vs CodaVax-RSV vaccinated volunteers. Numbers of SFU (spot forming units) were normalized against 10⁶ cells and graphed on Y axis.

FIG. 2 shows changes in numbers of SFU compared to the day 1 post vaccinations. Green line indicates 1.3× fold increase in SFU that we considered significant induction of T cell response.

FIG. 3 depicts the geometric mean fold rise in T-cell Response—Modified Intent-to treat Population. Abbreviations: FFU=focus-forming units; GMFR—geometric mean fold rise.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Deoptimized” as used herein with respect to a virus such as RSV refers to modified viruses in which their genome or anti-genome, in whole or in part, has synonymous codons and/or codon rearrangements and/or variation of codon pair bias. The substitution of synonymous codons alters various parameters, including for example, reducing codon bias, reducing codon pair bias, density of deoptimized codons and deoptimized codon pairs, RNA secondary structure, CpG dinucleotide content, C+G content, UpA dinucleotide content, translation frameshift sites, translation pause sites, the presence or absence of tissue specific microRNA recognition sequences, or any combination thereof, in the genome or anti-genome.

“Anti-genome” as used herein refers to the reverse complement of the actual viral genome sequence that would be present inside a virus particle. For example, RSV is a negative sense virus, it is common convention practice to show the genome in the anti-genome sense, which contains the protein coding sequences in the forward orientation, in which it would be read by the ribosome.

A “subject” as used herein means any animal or artificially modified animal. Animals include, but are not limited to, humans, non-human primates, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “prophylactically effective” is any amount of a vaccine or virus composition that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold, 25-fold, 50-fold, or 100 fold. For example, if a subject has a 1% chance of becoming infected with a virus, a two-fold reduction in the likelihood of the subject becoming infected with the virus would result in the subject having a 0.5% chance of becoming infected with the virus.

As used herein, a “therapeutically effective” is any amount of a vaccine or virus composition that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

CodaVax-RSV is based on a live attenuated RSV vaccine strain, designated as MinL4.0, created using the principle of Synthetic Attenuated Virus Engineering (SAVE), a synthetic biology method to “deoptimize” viral genes for slowed translation in the human host cell (Coleman 2008, Nouën 2017, incorporated herein by reference as though fully set forth). Attenuation is achieved by the large-scale recoding of viral genes using a computer algorithm, designed to replace preferred codons and codon pairs, with synonymous codons that are translated more slowly. This process is referred to as codon and codon-pair deoptimization (CPD). CPD creates hundreds of silent mutations that produce amino acid sequences 100% identical to those of the target virus yet attenuate the virus by orders of magnitude. The recoded virus is synthesized de novo. Although deoptimized viral genes encode the wildtype protein sequence, the CPD causes host cell ribosomes to produce less of the targeted proteins at a slower rate. As a result, the virus is significantly attenuated, but induces host cell immune responses that are very similar to wildtype infection. Since the sequences are modified by hundreds to thousands of such deoptimizing mutations, the viral attenuation phenotypes are genetically stable.

MinL4.0 (e.g., SEQ ID NO:1), the attenuated RSV strain contained in CodaVax-RSV, was synthesized with CPD of the RSV L gene plus 4 additional point mutations. These four additional sequence changes conferred increased genetic stability, further attenuation, and increased immunogenicity in animal models.

As such, methods of eliciting an immune response utilizing MinL4.0 and other similar deoptimized viruses in a subject are provided herein.

Various embodiments of the present invention provide for a method of eliciting an immune response in a subject, comprising intranasally administering one or more doses of an effective amount of a composition comprising a deoptimized respiratory syncytial virus (RSV) to the subject to elicit the immune response, wherein the effective amount is about 10³-10⁹ focus forming units (FFU) of the deoptimized RSV, wherein the immune response comprises IgA immune response, cellular immune response or both. In various embodiments, the effective amount is a prophylactically effective. In various embodiments, the effective amount is a therapeutically effective. In various embodiments, the method elicits a protective immune response.

In various embodiments, the method further comprises identifying the subject in need of eliciting the immune response to RSV prior to intranasally administering one or more doses of an effective amount of a composition comprising a deoptimized RSV.

In various embodiments, the immune response is an immune response to RSV. In various embodiments, the immune response is an IgA immune response, cellular immune response or both to RSV. In various embodiments, the immune response is a T-cell immune response to RSV.

In various embodiments, intranasally administering one or more doses of the effective amount of the composition comprises administering two doses, wherein a second dose is administered about 28 days after a first dose. In various embodiments, intranasally administering one or more doses of the effective amount of the composition comprises administering two doses, wherein a second dose is administered at least 28 days after a first dose. In various embodiments, intranasally administering one or more doses of the effective amount of the composition comprises administering two doses, wherein a second dose is administered 28-35 days after a first dose. In various embodiments, intranasally administering one or more doses of the effective amount of the composition comprises administering two doses, wherein a second dose is administered 21-49 days after a first dose.

In various embodiments, the method further comprises administering one or more booster doses one or more years after the initial dosing regimen. For example, 1 year, 2 years, 3 years, 4 years, 5 years, 10 years, 15 years, 20 years, 25 years, 30 years, 35 years, 40 years, 45 years, 50 years, 55 years, 60 years, 65 years, or 70 years. In various embodiments, the one or more booster dose is administered when the subject is older; for example, the subject was administered with the initial dosing regimen as a child, and a booster dose is administered when the subject is over 50 years old. In various embodiments, the subject is a human subject.

In various embodiments, intranasally administering comprises administering via nose drops. The nose drops can be given via a syringe to assist in proper dosing. In various embodiments, intranasally administering comprises administering via nasal spray.

In various embodiments, the effective amount is about 10⁴-10⁸ FFU of the deoptimized RSV. In various embodiments, the effective amount is about 10⁵-10⁷ FFU of the deoptimized RSV. For example, about 10⁴-10⁸ FFU or about 10⁵-10⁷ FFU of the deoptimized RSV can be an appropriate effective amount in an adult subject. In various embodiments, the effective amount is about 10³-10⁶ FFU of the deoptimized RSV. In various embodiments, the effective amount is about 10⁴-10⁵ FFU of the deoptimized RSV. For example, about 10³-10⁶ FFU or about 10⁴-10⁵ FFU of the deoptimized RSV can be an appropriate effective amount in a pediatric subject.

In various embodiments, the effective amount is about 2-3×10⁴-10⁸ FFU of the deoptimized RSV. In various embodiments, the effective amount is about 2-3×10⁵-10⁷ FFU of the deoptimized RSV. For example, about 2-3×10⁴-10⁸ FFU or about 2-3×10⁵-10⁷ FFU of the deoptimized RSV can be an appropriate effective amount in an adult subject. In various embodiments, the effective amount is about 2-3×10³-10⁶ FFU of the deoptimized RSV. In various embodiments, the effective amount is about 2-3×10⁴-10⁵ FFU of the deoptimized RSV. For example, about 2-3×10³-10⁶ FFU or about 2-3×10⁴-10⁵ FFU of the deoptimized RSV can be an appropriate effective amount in a pediatric subject.

In various embodiments, the effective amount is the amount of one dose.

In various embodiments, the effective amount is about 10⁵ FFU. In various embodiments, the effective amount is about 2×10⁵ FFU. In various embodiments, the effective amount is about 10⁶ FFU. In various embodiments, the effective amount is about 2×10⁶ FFU. In various embodiments, the effective amount is the amount of one dose. For example, if the effective amount is about 2×10⁶ FFU, when the deoptimized RSV is given as two doses, each dose will have about 2×10⁶ FFU.

In various embodiments, the effective amount is about 10³ FFU. In various embodiments, the effective amount is about 2×10³ FFU. In various embodiments, the effective amount is about 10⁴ FFU. In various embodiments, the effective amount is about 2×10⁴ FFU. In various embodiments, the effective amount is about 10⁵ FFU. In various embodiments, the effective amount is about 2×10⁵ FFU. In various embodiments, the effective amount is about 10⁶ FFU. In various embodiments, the effective amount is about 2×10⁶ FFU. In various embodiments, the effective amount is about 10⁷ FFU. In various embodiments, the effective amount is about 2×10⁷ FFU. In various embodiments, the effective amount is about 10⁸ FFU. In various embodiments, the effective amount is about 2×10⁸ FFU. In various embodiments, the effective amount is about 10⁹ FFU. In various embodiments, the effective amount is about 2×10⁹ FFU.

In various embodiments, one dose of the effective amount is provided in a volume of about 1 mL. In various embodiments, one dose of the effective amount is administered in about four aliquots. For example, if the effective amount is provided in a volume of 1 mL, each aliquot will be about 250 μL in volume. In various embodiments, one dose of the effective amount is administered in about 2 aliquots. In various embodiments, one dose of the effective amount is administered in about 6 aliquots.

In various embodiments, a first aliquot is administered in a first nostril, and a second aliquot is administered in a second nostril within 10 minutes of the first aliquot being administered in the first nostril, and a third aliquot and a fourth aliquot are administered in to the first nostril and the second nostril, in either order, about 5-20 minutes after the second aliquot is administered into the second nostril.

In various embodiments, a first aliquot is administered in a first nostril, and a second aliquot is administered in a second nostril within 5 minutes of the first aliquot being administered in the first nostril, and a third aliquot and a fourth aliquot are administered in to the first nostril and the second nostril, in either order, about 10-15 minutes after the second aliquot is administered into the second nostril.

In various embodiments, the cellular immune response comprises a development of a T-cell response. In various embodiments, the development of the T-cell response is in comparison to a reference level of response based on a placebo group. In various embodiments, the reference level of response can be the average level of the placebo group. In various embodiments, the reference level of response can be the median level of the placebo group.

In various embodiments, the development of the T-cell response is in comparison to a prevaccination timepoint for the subject. In various embodiments, the method elicits an immune response comprising an increase in T-cell response as compared to a prevaccination timepoint. In various embodiments, the increase T-cell response as compared to a prevaccination timepoint is a statistically significant increase.

In various embodiments, the effective amount is about 10⁶ FFU and eliciting the immune response comprises a statistically significant increase in T-cell response as compared to a prevaccination timepoint. In various embodiments, the effective amount is about 2×10⁶ FFU and eliciting the immune response comprises a statistically significant increase in T-cell response as compared to a prevaccination timepoint. In various embodiments, the effective amount is about 10⁵ FFU and eliciting the immune response comprises an increase in T-cell response as compared to a prevaccination timepoint. In various embodiments, the effective amount is about 2×10⁵ FFU and eliciting the immune response comprises an increase in T-cell response as compared to a prevaccination timepoint.

The prevaccination timepoint discussed herein can be a timepoint just before the first dose of the vaccination. For example, the T-cell response can be measured from a blood draw taken the same day but prior to the vaccination. The prevaccination timepoint discussed herein can be a timepoint such as about 1 day before vaccination, about 5 days before vaccination, about 7 days before vaccination, about 10 days before vaccination, about 14 days before vaccination, or more. The prevaccination timepoint discussed herein can be a timepoint such as 1-7 days before vaccination, 1-2 weeks before vaccination, 2-4 weeks before vaccination, 4-8 weeks before vaccination, 2-3 months before vaccination, 3-6 months before vaccination or more.

In various embodiments, an enzyme-linked immune absorbent spot (ELISpot) analysis of peripheral blood mononuclear cells (PBMCs) from the subject shows at least a 1.3 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject shows at least a 1.4 fold increase of spot forming units (SFU) between a baseline value and a highest value during a post vaccination timepoint. In various embodiments, an enzyme-linked immune absorbent spot (ELISpot) analysis of peripheral blood mononuclear cells (PBMCs) from the subject shows at least a 1.5 fold increase of spot forming units (SFU) between a baseline value and a highest value during a post vaccination timepoint. In various embodiments, wherein an enzyme-linked immune absorbent spot (ELISpot) analysis of peripheral blood mononuclear cells (PBMCs) from the subject shows at least a 1.6 fold increase of spot forming units (SFU) between a baseline value and a highest value during a post vaccination timepoint. In various embodiments, wherein an enzyme-linked immune absorbent spot (ELISpot) analysis of peripheral blood mononuclear cells (PBMCs) from the subject shows at least a 1.7 fold increase of spot forming units (SFU) between a baseline value and a highest value during a post vaccination timepoint.

In various embodiments, the method further comprises performing an enzyme-linked immune absorbent spot (ELISpot) analysis of peripheral blood mononuclear cells (PBMCs) from the subject to obtain a baseline value. In various embodiments, the method further comprises performing one or more an enzyme-linked immune absorbent spot (ELISpot) analysis of peripheral blood mononuclear cells (PBMCs) from the subject post vaccination.

In various embodiments, in reference a baseline, the baseline can be measured from a biological sample obtained prior to administration of the deoptimized RSV. For example, the biological sample for the baseline measurement is taken on the day of the first administration of the deoptimized RSV, prior to the administration. In other examples, a biological sample for the baseline measurement can be taken days or weeks prior to the day of the first administration of the deoptimized RSV. As an example, the biological sample for the baseline measurement can be taken as part of identifying the subject for eliciting the immune response, IgA immune response, cellular immune response, T-cell immune response.

In various embodiments, the effective amount is about 10⁶ FFU and eliciting the immune response comprises at least a 2.4 fold rise in IgA levels in the subject. In various embodiments, the effective amount is about 2×10⁶ FFU and eliciting the immune response comprises at least a 2.4 fold rise in IgA levels in the subject.

In various embodiments, the effective amount is about 10⁵ FFU and eliciting the immune response comprises at least a 1.1 fold rise in IgA levels in the subject. In various embodiments, the effective amount is about 2×10⁵ FFU and eliciting the immune response comprises at least a 1.1 fold rise in IgA levels in the subject.

In various embodiments, the effective amount is about 10⁵-10⁶ FFU and eliciting the immune response comprises at least a 1.3 fold rise in IgA levels in the subject. In various embodiments, the effective amount is about 2×10⁵ to 2×10⁶ FFU and eliciting the immune response comprises at least a 1.3 fold rise in IgA levels in the subject.

In various embodiments, the rises in IgA levels can be compared to the IgA levels the subject has prior to being administered the first dose of the deoptimized RSV. In other embodiments, the rises in IgA levels can be compared to the IgA levels the subject has prior to being administered a subsequent dose of the deoptimized RSV. In various embodiments, these rises in IgA levels is in mucosal IgA.

In various embodiments, the method further comprises analyzing IgA levels from the subject to obtain a baseline value. In various embodiments, the method further comprises analyzing IgA levels one or more times from the subject post vaccination.

In various embodiments, the deoptimized RSV comprises the genome or anti-genome having the sequence of SEQ ID NO:1. That is, the deoptimized RSV is encoded by the genome or anti-genome having the sequence of SEQ ID NO: 1.

In various embodiments, the deoptimized RSV comprises the genome or anti-genome having the sequence of SEQ ID NO:1 but with up to 12 amino acid substitutions, deletions or additions. In various embodiments, the deoptimized RSV comprises the genome or anti-genome having the sequence of SEQ ID NO:1 but with up to 9 amino acid substitutions, deletions or additions. In various embodiments, the deoptimized RSV comprises the genome or anti-genome having the sequence of SEQ ID NO:1 but with up to 6 amino acid substitutions, deletions or additions. In various embodiments, the deoptimized RSV comprises the genome or anti-genome having the sequence of SEQ ID NO:1 but with up to 3 amino acid substitutions, deletions or additions. In various embodiments, the deoptimized RSV comprises the genome or anti-genome having the sequence of SEQ ID NO:1 but with up to 2 amino acid substitutions, deletions or additions. In various embodiments, the deoptimized RSV comprises the genome or anti-genome having the sequence of SEQ ID NO:1 but with up to 1 amino acid substitution, deletion or addition.

In various embodiments, the deoptimized RSV comprises the genome or anti-genome of a deoptimized RSV as described by International Patent Application No. PCT/US2014/015274 (published as WO 2014/124238) herein incorporated by reference as though fully set forth. In various embodiments, the deoptimized RSV comprises the genome or anti-genome of a deoptimized RSV as described by International Patent Application No. PCT/US2017/053047 (published as WO 2018/057950) herein incorporated by reference as though fully set forth, for example, RSV Min_L-NPM2-1[N88K]L of WO 2018/057950.

In various embodiments, the subject is under the age of 18. In various embodiments, the subject is under the age of 17. In various embodiments, the subject is under the age of 13. In various embodiments, the subject is under the age of 7. In various embodiments, the subject is under the age of 6. In various embodiments, the subject is under the age of 2.

In various embodiments, the subject is 6 mo to 2 years old. In various embodiments, the subject is 3 years old to 5 years old. In various embodiments, the subject is 6 years old to 11 years old. In various embodiments, the subject is 12 years old to 17 years old.

In various embodiments, the subject is between 18-49 years old. In various embodiments, the subject is at least 50 years old. In various embodiments, the subject is at least 55 years old. In various embodiments, the subject is at least 60 years old.

Various embodiments provide for a method of eliciting an IgA immune response, cellular immune response or both in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 2×10⁵ focus forming units (FFU) of the deoptimized RSV, wherein the deoptimized RSV comprises the genome or anti-genome having the sequence of SEQ ID NO:1, and wherein an enzyme-linked immune absorbent spot (ELISpot) analysis of peripheral blood mononuclear cells (PBMCs) from the subject shows at least a 1.3 fold increase of spot forming units (SFU) between a baseline value and a highest value during a post vaccination timepoint. In various embodiments, the effective amount is a prophylactically effective. In various embodiments, the effective amount is a therapeutically effective. In various embodiments, the method elicits a protective immune response. Various embodiments of the present invention provide for a method of eliciting an IgA immune response, cellular immune response or both in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 2×10⁶ focus forming units (FFU) of the deoptimized RSV, and wherein the deoptimized RSV comprises the genome or anti-genome having the sequence of SEQ ID NO:1, and wherein eliciting the immune response comprises at least a 2.4 fold rise in IgA levels in the subject. In various embodiments, the effective amount is a prophylactically effective. In various embodiments, the effective amount is a therapeutically effective. In various embodiments, the method elicits a protective immune response.

In various embodiments, the method further comprises identifying the subject in need of eliciting an IgA immune response, cellular immune response or both prior to intranasally administering one or more doses of an effective amount of a composition comprising a deoptimized RSV.

In various embodiments, the method further comprises performing an enzyme-linked immune absorbent spot (ELISpot) analysis of peripheral blood mononuclear cells (PBMCs) from the subject to obtain a baseline value. In various embodiments, the method further comprises performing one or more an enzyme-linked immune absorbent spot (ELISpot) analysis of peripheral blood mononuclear cells (PBMCs) from the subject post vaccination.

In various embodiments, the method further comprises analyzing IgA levels from the subject to obtain a baseline value. In various embodiments, the method further comprises analyzing IgA levels one or more times from the subject post vaccination.

In various embodiments, the subject is under the age of 18. In various embodiments, the subject is under the age of 17. In various embodiments, the subject is under the age of 13. In various embodiments, the subject is under the age of 7. In various embodiments, the subject is under the age of 6. In various embodiments, the subject is under the age of 2.

In various embodiments, the subject is 6 mo to 2 years old. In various embodiments, the subject is 3 years old to 5 years old. In various embodiments, the subject is 6 years old to 11 years old. In various embodiments, the subject is 12 years old to 17 years old.

In various embodiments, the subject is between 18-49 years old. In various embodiments, the subject is at least 50 years old. In various embodiments, the subject is at least 55 years old. In various embodiments, the subject is at least 60 years old.

Various embodiments provide for a method of eliciting an T-cell immune response in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 10⁶ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint. In various embodiments, the increase is a statistically significant increase. In various embodiments, the effective amount is a prophylactically effective. In various embodiments, the effective amount is a therapeutically effective. In various embodiments, the method elicits a protective immune response. Various embodiments provide for a method of eliciting an T-cell immune response in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 2×10⁶ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint. In various embodiments, the increase is a statistically significant increase. In various embodiments, the effective amount is a prophylactically effective. In various embodiments, the effective amount is a therapeutically effective. In various embodiments, the method elicits a protective immune response.

Various embodiments provide for a method of eliciting an T-cell immune response in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 10⁵ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint. In various embodiments, the effective amount is a prophylactically effective. In various embodiments, the effective amount is a therapeutically effective. In various embodiments, the method elicits a protective immune response.

Various embodiments provide for a method of eliciting an T-cell immune response in a subject, comprising intranasally administering a first dose of an effective amount of a composition comprising a deoptimized respiratory syncytial virus (RSV) to the subject to elicit the immune response; intranasally administered a second dose of the effective amount of the composition comprising the deoptimized RSV 28-35 days after the first dose; wherein the effective amount is about 2×10⁵ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint. In various embodiments, the effective amount is a prophylactically effective. In various embodiments, the effective amount is a therapeutically effective. In various embodiments, the method elicits a protective immune response.

In various embodiments, the method further comprises identifying the subject in need of eliciting an T-cell immune response prior to intranasally administering one or more doses of an effective amount of a composition comprising a deoptimized RSV.

In various embodiments, the method further comprises performing an enzyme-linked immune absorbent spot (ELISpot) analysis of peripheral blood mononuclear cells (PBMCs) from the subject to obtain a baseline value. In various embodiments, the method further comprises performing one or more an enzyme-linked immune absorbent spot (ELISpot) analysis of peripheral blood mononuclear cells (PBMCs) from the subject post vaccination.

In various embodiments, the method further comprises analyzing T-cell immune response from the subject to obtain a baseline value. In various embodiments, the method further comprises analyzing T-cell immune response one or more times from the subject post vaccination.

In various embodiments, the subject is under the age of 18. In various embodiments, the subject is under the age of 17. In various embodiments, the subject is under the age of 13. In various embodiments, the subject is under the age of 7. In various embodiments, the subject is under the age of 6. In various embodiments, the subject is under the age of 2.

In various embodiments, the subject is 6 mo to 2 years old. In various embodiments, the subject is 3 years old to 5 years old. In various embodiments, the subject is 6 years old to 11 years old. In various embodiments, the subject is 12 years old to 17 years old.

In various embodiments, the subject is between 18-49 years old. In various embodiments, the subject is at least 50 years old. In various embodiments, the subject is at least 55 years old. In various embodiments, the subject is at least 60 years old.

The prevaccination timepoint discussed herein can be a timepoint just before the first dose of the vaccination. For example, the T-cell response can be measured from a blood draw taken the same day but prior to the vaccination. The prevaccination timepoint discussed herein can be a timepoint such as about 1 day before vaccination, about 5 days before vaccination, about 7 days before vaccination, about 10 days before vaccination, about 14 days before vaccination, or more. The prevaccination timepoint discussed herein can be a timepoint such as 1-7 days before vaccination, 1-2 weeks before vaccination, 2-4 weeks before vaccination, 4-8 weeks before vaccination, 2-3 months before vaccination, 3-6 months before vaccination or more.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The following clinical trial protocol was used to generate the data described in Examples 2 and 3. While this protocol contains inclusion criteria, exclusion criteria, and medication, contraception and other restrictions, and the like, these criteria and restrictions are not to be interpreted as limitations to the practice of the invention as claimed, unless it/they is/are specifically noted to be excluded in the claims. Indeed, certain individuals excluded from being eligible from this study are contemplated as individuals that can and should receive the methods described herein.

CodaVax-RSV is a sterile solution delivered by IN administration. For this FIH Phase I clinical trial in healthy older adults, two nominal dose levels of CodaVax-RSV at approximately 2×10⁵ and 2×10⁶ FFU per dose, are administered 28 days apart (i.e., on Day 1 and Day 29). Subjects will be randomized 2:1 to CodaVax-RSV or normal saline placebo within each group. The dose levels to be explored are described in Table 1.

Decisions to continue dosing after sentinel dosing and escalate the dose to the higher dose cohort will be dependent upon review of emerging safety data by the SMC. The PI will review the safety data from the first day of dosing for the first 2 sentinels and advise the rest of the SMC before dosing the remainder of the sentinel cohort. The SMC will then review the blinded safety data through Day 8 for all sentinel participants and low dose cohort participants before randomizing subsequent participants in each group and will review the available blinded safety data before participants in each cohort begin receiving their second dose.

TABLE 1
CodaVax-RSV Dose Levels

| | Dose Level | Age | Participant |
|---|---|---|---|
| Dosing Group | (FFU) | (years) | Count (n = 42) |
| Low Dose Sentinel Group 1 | 2 × 10⁵ | 18-49 | 2 |
| Low Dose Sentinel Group 2 | 2 × 10⁵ | 18-49 | 4 |
| Low Dose Group | 2 × 10⁵ | 50-75 | 15 |
| High Dose Group | 2 × 10⁶ | 50-75 | 21 |

The study will be conducted healthy male and female volunteers aged 18 to 49 years (inclusive at the time of informed consent) for the sentinel groups and 50 to 75 years (inclusive at the time of informed consent) for the main dosing groups.

Women of childbearing potential will be included and are subject to contraceptive requirements during the study from Screening through 6 months after the second vaccination. Women of childbearing potential must demonstrate negative pregnancy testing at Screening and before administration of study drug. This is in line with regulatory Guidance on Nonclinical Safety Studies for the Conduct of Human Clinical Trials and Marketing Authorization for Pharmaceuticals (United States Food and Drug Administration [FDA] 2006).

### Study Objectives

| | |
|---|---|
| OBJECTIVES | ENDPOINTS |

Primary Objective

| | |
|---|---|
| To assess the safety and tolerability of | Reactogenicity events: counts and |
| CodaVax-RSV at nominal doses of | percentages of participants with local and |
| approximately 2 × 10⁵ or 2 × 10⁶ FFU given | systemic events along with symptom |
| IN in a regimen of two doses 28 days apart | severity and duration assessed for 7 days |
| | after vaccination |
| | AEs: counts and percentages of |
| | participants with AEs not included as |
| | reactogenicity events from Day 1 through |
| | 57 |
| | MAEs, NCIs, and SAEs will be captured |
| | from Day 1 until Day 209 |

Secondary Objectives

| | |
|---|---|
| To assess the humoral immunogenicity of | RSV-specific IgG antibodies measured by |
| CodaVax-RSV given IN in a regimen of | ELISA in serum on Days 1, 15, 29, 43, 57, |
| two doses 28 days apart | 113, and 209 |
| | GMT |
| | GMR of post-vaccination titers against |
| | baseline |
| | Seroconversion rate (percent of |
| | participants with at least 4-fold increase |
| | over baseline) |
| | Neutralizing antibodies in serum measured |
| | via RSV micro neutralization assay in |
| | serum on Days 1, 15, 29, 43, 57, 113, and |
| | 209 |
| | GMT |
| | GMR against baseline |
| | Seroconversion rate (Percent of |
| | participants with at least 4-fold increase |
| | over baseline) |
| | RSV-specific IgA antibodies measured by |
| | ELISA in NP swab specimens on Days 1, |
| | 15, 29, 43 and 57 |
| | GMT |
| | Seroconversion rate (percent of |
| | participants with at least 4-fold increases |
| | over baseline |

Exploratory Objectives

| | |
|---|---|
| To assess cellular immunogenicity of | T cell responses as measured by: |
| CodaVax-RSV | RSV-stimulated PBMC IFN-γ |
| | ELISPOT on Days 1 and 8, 29, and 36 |
| To assess the impact of baseline RSV | Subgroup analysis of immunogenicity |
| serostatus on immunogenicity of CodaVax- | measured by baseline RSV neutralizing |
| RSV | antibody titer |
| To assess incidence of respiratory illness | Counts and percentages of participants |
| including that due to RSV infection | with AEs consistent with respiratory |
| following vaccination | infection |
| | Respiratory virus PCR panel for |
| | symptomatic respiratory infection |
| To assess the genetic stability of Coda Vax- | Sequence analysis of RSV isolates |
| RSV | |
| To assess the magnitude and duration of | Shedding measured by qPCR of |
| vaccine shedding | nasopharyngeal swabs |
| To assess the risk of vaccine-related viremia | Vaccine viremia measured by qPCR of |
| | serum and confirmed with virus culture |

### Overall Study Design

This is a Phase 1, randomized, double-blind, placebo-controlled study of the safety, tolerability and immunogenicity of two single IN CodaVax-RSV vaccinations administered 28 days apart. The study include 2 sentinel groups of a total of 6 younger adults aged 18 to 49 years followed by approximately 36 healthy older adults with low baseline RSV titers aged 50 to 75 years.

Eligible participants are randomized 2:1 to CodaVax-RSV or Placebo (normal saline) in two dose escalating cohorts. All participants will receive two doses, 28 days apart. The low dose cohort receives nominal doses of approximately 2×10⁵ FFU and the high dose cohort receives nominal doses of approximately 2×10⁶ FFU administered IN in a total volume of 1 mL given as 250 μL each nostril repeated after 10 to 15 minutes. The dose levels and dosing for this study will be as follows (Table 2)

TABLE 2
Dose Groups and Participant Counts

| | Dose | | Participant | CodaVax- | |
|---|---|---|---|---|---|
| | Level | Age | Count | RSV | Placebo |
| Dosing Group | (FFU) | (years) | (n = 42) | n = 28 | n = 14 |
| Low Dose | 2 × 10⁵ | 18-49 | 2 | 1 | 1 |
| Sentinel Group 1 | | | | | |
| Low Dose | 2 × 10⁵ | 18-49 | 4 | 3 | 1 |
| Sentinel Group 2 | | | | | |
| Low Dose Group | 2 × 10⁵ | 50-75 | 15 | 10 | 5 |
| High Dose Group | 2 × 10⁶ | 50-75 | 21 | 14 | 7 |

After providing informed consent, participants who appear eligible will be screened by routine physical examination, ECG and clinical laboratory testing (hematology, biochemistry, and urinalysis) performed no more than 28 days prior to randomization to determine eligibility. Older adult participants will be recruited from a previous screening study designed to identify those with low baseline RSV antibody titers. On Day 1, after physical examination, blood draw for PBMC and serum antibody, and NP swabs for mucosal antibody, eligible participants will be randomized to either active treatment or placebo within each dosing group, with dosing groups defined as in table 6 above, using a block randomization scheme provided by the unblinded statistician to the site pharmacist. Participants will be administered the first of two vaccines and will be observed for 2 hours post-dose and will be discharged following successful completion of all specified assessments.

Participants will complete a daily diary recording oral temperature and presence and severity of solicited symptoms for 7 days after each vaccination. Participants will return for targeted physical examination, serum collection, NP swabs and AE and concomitant medication review in accordance with the Schedule of Assessments. On Day 8 safety laboratory studies and PBMCs will also be collected. Participants will receive their second vaccination

28 days after the initial vaccination (Day 29). Participants will be observed for 2 hours post-vaccination and will be discharged following successful completion of all specified assessments. Study procedures on Days 32, 36, and 43 will be similar to those following the first vaccination. On Day 57 participants will undergo similar assessments with the addition of a follow-up pregnancy test and ECG.

Oversight for the study will be provided by an SMC comprised of the Investigator, the Sponsor's MM and an Independent MM. The PI will review the safety data from the first day of dosing for the first 2 sentinels and advise the rest of the SMC before dosing the remainder of the sentinel cohort. The SMC will then review the blinded safety data through Day 8 for all sentinel participants and the low dose group before randomizing participants to subsequent groups and will review the available blinded safety data before participants in each cohort begin receiving their second dose. Two late follow-up visits are scheduled after Day 57.

Participants who develop symptoms of respiratory infection during the late follow-up period will have a specimen obtained for respiratory virus PCR panel (followed by confirmatory sequencing if positive for RSV), which may be during an unscheduled visit. Clinically significant laboratory abnormalities may also be repeated at unscheduled visits. All AEs and concomitant medications will be recorded through Day 57, and MAEs, NCIs, SAEs, and concomitant medications of special interest (vaccines and immunosuppressive medications) will be recorded from Day 1 through 209 (EOS). Study visits and assessments will occur as delineated in the Schedule of Assessments.

### Number of Participants

Approximately 42 participants will be randomized.

### Treatment Assignment

### Randomization

Clinical Network Services will prepare a Request for Randomization Schedule and manage the randomization of eligible study participants as per this document. A randomization list will be prepared using a statistical software package by a NVT Biostatistician.

Each participant will be provided with a unique screening number post-documentation of informed consent. Once deemed eligible for enrollment in the study, the participant will be assigned a sequential randomization number prior to first dosing. Participants who withdraw from the study, for any reason, without completing all necessary screening assessments will be considered screen failures.

The details of randomization will be further documented in the Request for Randomization Schedule.

### Safety Monitoring Committee

Oversight will be provided by an SMC including, at a minimum, the PI, the Sponsor's MM and an Independent MM who will conduct a review of the blinded safety data from the sentinel groups prior to randomization of the remainder of participants in the low dose cohort, dose escalation to the high dose cohort, and before participants are administered their second vaccination in each group.

Following each review, the SMC will make the following recommendations:
To continue the study as planned
To continue the study with modifications
To temporarily suspend or terminate the study
If a decision is made to temporarily suspend or terminate the study, the relevant SMC minutes will be forwarded to the HREC.

Stopping Criteria

If any of the following stopping rules are met, randomization and dosing will stop until the SMC conduct blinded safety data review and assess relatedness and risk to subsequent study participants.
Possibly related SAE
Grade 4 laboratory value or AE
Two or more similar Grade 3 events or laboratory abnormalities Minutes of such blinded reviews will be forwarded to the HREC.

### Criteria for Study Termination

The study will be completed as planned unless:
New information or other evaluation regarding the safety of the study medication indicates a change in the known risk/benefit profile for the compound, such that the risk/benefit is no longer acceptable for participants participating in the study. This may be determined by the Sponsor, the Investigator, the local MM, the SMC, the HREC or regulatory authorities.
The study is terminated by the Sponsor for administrative reasons.
If the Sponsor, the HREC, or regulatory authority elects to terminate or suspend the study or the participation of the investigational site, a study-specific procedure for early termination or suspension will be provided by the Sponsor. The procedure will be followed by the investigational site during termination or study suspension.

### Participant Inclusion Criteria

To be eligible for this study, a participant has to meet all of the following inclusion criteria:

1. Healthy men and women aged 50 to 75 years of age, inclusive (at the time of informed consent) for the main dosing group or healthy male and female volunteers aged 18 to 49 years of age, inclusive (at the time of informed consent) for the sentinel groups;

2. BMI≥18.0 kg/m2 and ≤35 kg/m2;

3. Participants must be willing to comply with the following conditions to prevent the spread of GMOs according the OGTR License (DIR 161):
a. Hygiene measures intended to prevent interpersonal transmission of study drug must be implemented, including but not limited to frequent handwashing with soap or hand disinfectant, respiratory hygiene and cough etiquette within 14 days after each vaccination
b. Blood, tissue or organs must not be donated from the time of their first vaccination until 6 months after their last vaccination
c. Contact is to be avoided with Excluded persons*, children aged 2 years and younger and residents of aged care facilities within 14 days after each vaccination
d. All tissues and materials used to collect respiratory secretions for 14 days after each vaccination are to be sealed in a primary container and placed within a secondary container so that it is not accessible to children or animals until it is returned to the study site for disposal

*Excluded persons means women who are or may be pregnant; persons with immunodeficiency or immunosuppression that result in a full or partial impairment of the immune system.

4. Adequate venous access for repeated phlebotomies;

5. Screening laboratory results within the normal range or Grade 1 abnormality if the Investigator documents clinical insignificance. Creatine kinase or bilirubin may be Grade 2 if associated with normal ALT and AST and the Investigator considers the result not to be clinically significant due to vigorous exercise or Gilbert's syndrome;

6. Negative drug and alcohol screen at Screening (unless explained by prescribed medication);

7. For participants in the main dosing groups, eligible RSV neutralizing antibody titer as defined in the previous seroepidemiology study;

8. Women of childbearing potential must be non-pregnant and non-lactating, and must use an acceptable, highly effective double barrier contraception from Screening until 6 months after the final vaccination. Double contraception is defined as a condom AND one other form of the following:
Established hormonal contraception (OCPs, long-acting implantable hormones, injectable hormones);
A vaginal ring or an IUD;
Documented evidence of surgical sterilization at least 6 months prior to screening (e.g., tubal occlusion, hysterectomy, bilateral salpingectomy, or bilateral oophorectomy for women or vasectomy for men [with appropriate post-vasectomy documentation of the absence of sperm in semen] provided the male partner is a sole partner)
   Women not of childbearing potential must be postmenopausal for >12 months. Postmenopausal status will be confirmed through testing of FSH levels≥40 IU/mL at Screening for amenorrhoeic female participants. Females who are abstinent from heterosexual intercourse will also be eligible.
      Periodic abstinence (e.g., calendar, ovulation, symptothermal, post-ovulation methods) and withdrawal are not considered highly effective methods of birth control.
      Female participants who are in same sex relationships are not required to use contraception.
      WOCBP must have a negative pregnancy test at Screening and Day 1 and be willing to have additional pregnancy tests as required throughout the study;
Males must be surgically sterile (>30 days since vasectomy with no viable sperm), abstinent, or if engaged in sexual relations with a WOCBP, his partner must be surgically sterile (e.g., tubal occlusion, hysterectomy, bilateral salpingectomy, bilateral oophorectomy) or using an acceptable, highly effective double barrier contraceptive method from Screening until 6 months after the final vaccination. Acceptable methods of double barrier contraception include the use of condoms and the use of an effective contraceptive for the female partner that includes: OCPs, long-acting implantable hormones, injectable-hormones, a vaginal ring or an IUD. Participants with same sex partners (abstinence from penile-vaginal intercourse) are eligible when this is their preferred and usual lifestyle.

9. Males must not donate sperm for at least 6 months after the final vaccination;

10. Participants must have the ability and willingness to attend the necessary visits to the CRU;

11. Participants must be willing and able to provide written informed consent prior to the commencement of any study procedures.

### Participant Exclusion Criteria

A participant who meets any of the following exclusion criteria must be excluded from the study:

1. Pregnant or lactating at Screening or planning to become pregnant (self or partner) prior to 6 months after the final vaccination;

2. Household contacts or caregivers of children<2 years of age or immunocompromised individuals (for the period up through 14 days post final vaccination). Immunocompromised individuals defined as but not limited to:
a. Persons who are HIV-infected
b. Persons who have received chemotherapy within 6 months
c. Persons receiving immunosuppressive agents
d. Person living with solid organ or bone marrow transplant;

3. Positive result for HIV, HBV, or HCV at Screening;

4. Asthma or other chronic lung disease that is greater than mild in severity. Specifically excluded are participants with any of the following history related to asthma or lung disease:
a. Daily symptoms for more than 1 week in the past 5 years;
b. Use of short acting beta 2 agonists in the past 5 years (e.g., albuterol);
c. Use of inhaled steroids, theophylline or pulse systemic steroids in the past 5 years; or
d. Any history of intubation or hospitalization related to asthma or other chronic lung disease.

5. History of diabetes mellitus (gestational diabetes is allowed if treatment was not required postpartum and serum glucose is currently within the normal range);

6. History of coronary artery disease, arrhythmia, or congestive heart failure;

7. Clinically significant ECG abnormality as determined by the Investigator at Screening;

8. Poorly controlled hypertension (systolic blood pressure>150 mmHg or diastolic blood pressure>95 mmHg) at Screening or predose on Day 1; 9. History of anaphylaxis or angioedema;

10. History of severe reaction to immunization;

11. Known allergy to any of the ingredients in the vaccine formulation;

12. History of chronic rhinitis, nasal septal defect, cleft palate, nasal polyps, or other nasal abnormality that might alter nasal mucosa and affect vaccine response;

13. Previous nasal surgery or nasal cauterization;

14. Epistaxis (nosebleed), symptoms of upper respiratory infection or subjective fever or malaise within 3 days prior to dosing on Day 1 (temporary exclusion criterion);

15. Any symptoms or signs on Day 1 that could inhibit the proper administration of the IP or interpretation of solicited AEs diary (e.g., temperature>38° C., nasal congestion or rhinorrhea) (temporary exclusion criteria);

16 Known or suspected malignancy, except for non-melanoma skin cancers and other early stage surgically excised malignancies that the Investigator considers to be exceedingly unlikely to recur;

17. Immunodeficiency including the use of corticosteroids (including IN steroids), alkylating drugs, antimetabolites, radiation, immune-modulating biologics, or other immunomodulating therapies within 90 days prior to dosing on Day 1, or for those participants that plan to use any of these during the study follow-up period. Topical steroid creams used on the skin are allowed;

18. History of bleeding disorder (e.g., factor deficiency, coagulopathy, or platelet disorder requiring special precautions);

19. History of autoimmune or demyelinating illness;

20. History of psychosis, hospitalization for psychiatric illness, or suicide attempt in the past 10 years;

21. History of seizures (other than childhood febrile seizures), dementia or progressive neurological disease;

22. Receipt of IN medications (including OTC medications but excluding saline) within 30 days prior to Day 1;

23. Receipt of any other IP within 30 days or 5 half-lives (whichever is longer) prior to Day 1;

24. Receipt of any vaccine, TB skin test or allergy antigen inoculations within 30 days prior to Day 1 or planned up to Day 57;

25. Receipt of IN vaccine within 90 days prior to Day 1;

26. Receipt of any licensed or investigational RSV vaccine;

27. Receipt of blood transfusion or blood products within 90 days prior to Day 1 or planned up through Day 57;

28. Planned elective hospitalization or surgical procedure through Day 57;

29. Other chronic medical conditions not mentioned must be stable without necessitating medication changes within 30 days prior to Day 1;

30. Past regular use or current use of IN illicit drugs;

31. Smokers of any type (e.g., cigarettes, electronic cigarettes, marijuana). Prior smokers must have quit smoking at least 30 days prior to Day 1;

32. Residents of aged care facilities

33. Employee of Codagenix, vendors, or research sites associated with the study;

34. Any medical, psychiatric or social condition, or occupational or other responsibility that, in the judgment of the Investigator, would interfere with or serve as a contraindication to protocol adherence, assessment of safety (including reactogenicity), or a participant's ability to give informed consent.

35. Positive result for COVID-19 PCR test within 28 days, or contact with known or suspected COVID-19 infected person, or travel, within 14 days prior to randomization, to area with endemic COVID-19 transmission in the community

### Medication, Contraception and Other Restrictions

### Medication

The following medications are prohibited through Day 57:
Systemic, intranasal or inhaled steroids
Systemic, intranasal, or inhaled immunosuppressives
Intranasal vaccines or medications (other than saline)
Vaccines of any type or skin testing for TB or allergies
Blood transfusion or immunoglobulin treatment
Any other IP

Note: Antipyretics and analgesics may be used to manage fever or other symptoms after daily recording of solicited events and temperature and will be recorded as concomitant medications but should not be given prophylactically.

Investigational vaccines or other biologic products for RSV are prohibited throughout the study.

### Immunosuppressive Medications

Immunosuppressive medications (including, but not limited to, the following list) are prohibited from 90 days before Day 1 to the end of the study. The following immunosuppressive concomitant medications are considered to be of special interest for the purposes of this study:

### Corticosteroids

prednisone (Deltasone, Orasone)
budesonide (Entocort EC)
prednisolone (Millipred)

### Calcineurin Inhibitors

cyclosporine (Neoral, Sandimmune, SangCya)
tacrolimus (Astagraf XL, Envarsus XR, Prograf)
   Mechanistic Target of Rapamycin (mTOR) Inhibitors
sirolimus (Rapamune)
everolimus (Afinitor, Zortress)

### Inosine Monophosphate Dehydrogenase (IMDH) Inhibitors

azathioprine (Azasan, Imuran)
leflunomide (Arava)
mycophenolate (CellCept, Myfortic)

### Biologics

abatacept (Orencia)
adalimumab (Humira)
anakinra (Kineret)
certolizumab (Cimzia)
etanercept (Enbrel)
golimumab (Simponi)
infliximab (Remicade)
ixekizumab (Taltz)
natalizumab (Tysabri)
rituximab (Rituxan)
secukinumab (Cosentyx)
tocilizumab (Actemra)
ustekinumab (Stelara)
vedolizumab (Entyvio)

### Monoclonal Antibodies

basiliximab (Simulect)
daclizumab (Zinbryta)
muromonab (Orthoclone OKT3)

### Additional Concomitant Medications of Interest

The following medications will also be considered concomitant medications of special interest for the purposes of this protocol:
Vaccines
Blood products
Immunoglobulin products

### Smoking

Smokers of any type (e.g., cigarettes, electronic cigarettes, marijuana) are excluded from this study. Prior smokers must have quit smoking at least 30 days prior to Day 1.

### Contraception

Contraceptive requirements are as discussed in this example.

### Description of Study Drug

### CodaVax-RSV

CodaVax-RSV is composed of a single-stranded, negative-sense RNA [(−)ssRNA]generated by CPD open reading frames (ORFs) designed using computational algorithms (Coleman 2008, Mueller 2010), and is based on RSV sequence M74568 (biological wt RSV strain A2) (Nouën 2017).

CodaVax-RSV will be delivered IN via syringe (no needle). The vialled vaccine will be thawed and loaded into the syringe by the pharmacy at the clinic (in a Class II biosafety cabinet), prior to IN administration. The lower study dose will be achieved by dilution onsite with Leibovitz's L-15 Medium. Further details will be outlined in a Pharmacy Manual.

### Placebo

The placebo in this study will be normal saline delivered IN via syringe (no needle).

### Concomitant Medications

All medications, including OTC medications, vitamins, and herbal supplements, taken during the 30 days prior to Screening will be recorded and reviewed by the Investigator to determine whether the participant is suitable for inclusion in the study.

The use of any IP or investigational medical device within 30 days prior to Screening is prohibited.

Prior therapy or concomitant therapy (after study drug administration) with any medications, including both prescription and non-prescription drugs should be discussed with the Investigator and Sponsor's MM before study drug administration, except in the case of necessary treatment of AEs or where appropriate medical care necessitates that therapy should begin before the Investigator can consult with the Independent MM.

Paracetamol/acetaminophen (1-2 therapeutic doses per week) may be used for minor ailments during the course of the study, at the discretion of the Investigator, without prior consultation with Sponsor's MM.

All medications, including OTC medications, vitamins, and herbal supplements, taken by participants through Day 57 will be recorded in the eCRF and coded using the most current WHO drug dictionary available at NVT. After Day 57, only concomitant medications of special interest will be recorded. Prior and concomitant medications will be listed by participant and summarized by anatomical therapeutic chemical (ATC) and PT.

### Blinding

Blinding procedures for the study will be described in detail in the Data Management Plan and Pharmacy Manual.

The Sponsor, Investigator, MM, study personnel, and participants are not to make any effort to determine which therapy is being received. Unblinded pharmacy (or other qualified site) personnel will be used in this study to prepare the study drug.

Only in the case of an emergency, when knowledge of the study drug is essential for the clinical management or welfare of a specific participant, may the Investigator unblind a participant's treatment assignment. Copies of the randomization sequence and treatment codes will be kept in the pharmacy at the investigational site. If required, the Investigator may obtain a participant's treatment assignment from a member of the unblinded pharmacy staff. However, prior to any unblinding, the Investigator is strongly advised to discuss options with the MM, Sponsor or appropriate study personnel. The Investigator will record in source documentation, the date and reason for revealing the blinded treatment assignment for that participant and the names and roles of personnel unblinded.

As soon as possible, and without revealing the participant's study treatment assignment (unless important to the safety of participants remaining in the study), the Investigator must notify the Sponsor if the blind is broken for any reason and the Investigator was unable to contact the Sponsor prior to unblinding.

If the Investigator considers an AE to be of such severity as to require immediate specific knowledge of the identity and dose of the relevant product, the Investigator may break the study code for that participant.

All unblinding events must be promptly reported to the MM, the Sponsor, and CNS.

Following the Day 57 visit, a soft lock will be placed on the database, at which point the statistical team will be unblinded to produce interim tables and figures. The investigational site, study monitor, medical monitor, and Sponsor will remain blinded to individual treatment assignment through to the end of study.

### Study Drug Storage

Upon receipt, the study drug must be stored at −80° C.±10° C.

The filled syringes should be stored at 5±3° C. for no longer than 4 hours before use and transported to the clinic on ice.

The Investigator or designee will be fully responsible for the security, accessibility and storage of the study drug while it is at the investigational facility.

### Study Drug Preparation

Procedures relating to study drug preparation and dispensing are outlined in the Pharmacy Manual.

### Administration

The Investigator or designee is responsible for the training of study staff and participants as to the correct administration of the study drug. Doses will be administered in a volume of 1 mL given as 250 μL in each nostril repeated after 10-15 minutes of the second nostril being dosed. The second nostril must be dosed within 5 minutes of the first nostril, at each application.

### Study Schedule

Where possible, assessments scheduled for the same assessment time point should be conducted in order of least invasive to most invasive.

### After Vaccination

Participants will remain in the CRU to the completion of all scheduled post-dose procedures. The following procedures will be conducted post-dose:
Focused physical examination at 2 hours (±30 minutes) post-dose.
Vital signs at 2 hours (±30 minutes) post-dose (systolic and diastolic blood pressure, respiratory rate, pulse rate, and oral temperature) (after participant has been resting in a supine position for at least 5 minutes)

Note: AEs and concomitant medications will be continually monitored until the participant is discharged from the CRU.

Participants will be issued diary cards and thermometers and will be given instructions as to how to record oral temperature and reactogenicity events.

Participants will be discharged at approximately 2 hours post-dose and following satisfactory completion of all assessments and procedures.

### Follow-up Telephone Call (Day 2)

Participants will be contacted by site staff. Site staff will diary and ask about any AEs and concomitant medication use. Participants reporting grade 3 symptoms of respiratory virus infection will be asked to attend an unscheduled visit and provide an NP swab for PCR.

### Follow-Up (Days 4 and 8 [±1 Day])

Participants will return to the CRU for follow-up visits on Days 4 and 8 (+1 day).

The following procedures will be conducted at each of the follow-up visits unless otherwise indicated:
Focused physical examination
NP swabs for evaluation of vaccine shedding
Vital signs (systolic and diastolic blood pressure, respiratory rate, pulse rate, and oral temperature) (after participant has been resting in a supine position for at least 5 minutes)
Collect blood for clinical laboratory testing (hematology, biochemistry) (Day 8 only)
Collect blood for evaluation of viremia in serum
Collect blood for evaluation of T cell responses in PBMC (Day 8 only)
Review diary for reactogenicity events
Review of other AEs and concomitant medications
Diary cards will be collected at Visit 4 (Day 8 [±1 day]).

### Follow-Up (Day 15 [±3 Days])

Participants will return to the CRU for a follow-up visit on Day 15 (±3 days). The following procedures will be conducted at the follow-up visit:
Focused physical examination
NP swabs for evaluation of vaccine shedding and humoral immunogenicity (IgA antibodies)
Collect blood for evaluation of immunogenicity (antibodies in serum)
Review of other AEs and concomitant medications

### Day of Second Vaccination (Day 29 [±3 Days])

Before Vaccination

Participants will attend the CRU and the following assessments will be carried out within 2 hours prior to the administration of study drug unless otherwise noted:
Review to determine if the participant continues to be eligible to participate in the study
Review of other AEs and concomitant medications
Urine drug screen and alcohol breath test
Urine pregnancy test (WOCBP only)
Urinalysis
NP swabs for evaluation of vaccine shedding and humoral immunogenicity (IgA antibodies)
Vital signs (systolic and diastolic blood pressure, respiratory rate, pulse rate, and oral temperature) (after participant has been resting in a supine position for at least 5 minutes)
Collect blood for clinical laboratory testing (hematology, biochemistry)
Collect blood for evaluation of immunogenicity (antibodies in serum)
Collect blood for evaluation of T cell responses in PBMC

### Vaccination

Participants will be administered study drug in accordance with their assigned treatment. Study drug will be administered by assigned clinical staff.

### After Vaccination

Participants will remain in the CRU to the completion of all scheduled post-dose procedures. The following procedures will be conducted post-dose:
Focused physical examination at 2 hours (±30 minutes) post-dose
Vital signs at 2 hours (±30 minutes) post-dose (systolic and diastolic blood pressure, respiratory rate, pulse rate, and oral temperature) (after participant has been resting in a supine position for at least 5 minutes)

Note: AEs and concomitant medications will be continually monitored until the participant is discharged from the CRU.

Participants will be issued new diary cards and will be given instructions as to how to record oral temperature and reactogenicity events.

Participants will be discharged at approximately 2 hours post-dose and following satisfactory completion of all assessments and procedures.

### Follow-up Telephone Call (Day 30)

Participants will be contacted by site staff. Site staff will ask about any AEs and concomitant medication use. Participants reporting grade 3 symptoms of respiratory virus infection will be asked to attend an unscheduled visit and provide an NP swab for PCR.

### Follow-Up (Days 32 and 36 [±1 Day])

Participants will return to the CRU for follow-up visits on Days 32 and 36 (±1 day).

The following procedures will be conducted at each of the follow-up visits unless otherwise indicated:
Focused physical examination
NP swabs for evaluation of vaccine shedding
Vital signs (systolic and diastolic blood pressure, respiratory rate, pulse rate, and oral temperature) (after participant has been resting in a supine position for at least 5 minutes)
Collect blood for clinical laboratory testing (hematology, biochemistry) (Day 36 only)
Collect blood for evaluation of viremia in serum
Collect blood for evaluation of T cell responses in PBMC (Day 36 only)
Review diary for reactogenicity events
Review of other AEs and concomitant medications Diary cards will be collected at Visit 8 (Day 36 [±1 day]).

### Follow-Up (Day 43[±2 Days])

Participants will return to the CRU for a follow-up visit on Day 43 (±2 days). The following procedures will be conducted at the follow-up visit:
Focused physical examination.
NP swabs for evaluation of vaccine shedding and humoral immunogenicity (IgA antibodies)
Collect blood for evaluation of immunogenicity (antibodies in serum)
Review of other AEs and concomitant medications

### Follow-Up (Day 57 [±3 Days])

Participants will return to the CRU for a follow-up visit on Day 57 (+3 days from 28 days after dosing at Visit 6).

The following procedures will be conducted at the follow-up visit:
Urine pregnancy test (WOCBP only)
Urinalysis
Vital signs (systolic and diastolic blood pressure, respiratory rate, pulse rate, and oral temperature) (after participant has been resting in a supine position for at least 5 minutes)
Focused physical examination.
NP swabs for evaluation of vaccine shedding and humoral immunogenicity (IgA antibodies)
Collect blood for clinical laboratory testing (hematology, biochemistry)
Collect blood for evaluation of immunogenicity (antibodies in serum)
Review of other AEs and concomitant medications
12-lead ECG (after participant has been resting in a supine position for at least 5 minutes)

### Follow-Up (Day 113 [±10 Days])

Participants will return to the CRU for a follow-up visit on Day 113 (±10 days from 84 days after Visit 6).

The following procedures will be conducted at the follow-up visit:
Review of MAEs, NCIs, and SAEs only
Review of concomitant medications of special interest
NP swabs for evaluation of vaccine shedding
Collect blood for evaluation of immunogenicity (antibodies in serum) End of Study (Day 209 [±10 days])

Participants will return to the CRU for an EOS follow-up visit on Day 209 (±10 days from 6 months after dosing at Visit 6).

The following procedures will be conducted at the follow-up visit:
Urine pregnancy test (WOCBP only)
Collect blood for evaluation of immunogenicity (antibodies in serum)
Review of MAEs, NCIs, and SAEs only
Review of concomitant medications of special interest. This visit marks the end of participation in this study.

### Immunogenicity Assessments

### Serum Sample Collection and Analysis

Blood samples for serum will be collected, processed, and shipped for analysis in accordance with the site's standard operating procedures and the Laboratory Manual prior to dosing and at the time points delineated in the Schedule of Assessments. Serum will be separated within 2 hours and frozen at −80° C. (±10° C.).

Serum will be shipped frozen to the Codagenix laboratory in Farmingdale NY USA, and tested as follows:

### RSV Immunoglobulin G

RSV-specific serum IgG antibodies will be analyzed by ELISA, using RSV-A2 whole virus lysate as target antigen. Briefly, heat inactivated serum samples will be serially diluted and reacted with assay wells coated with RSV antigen. Bound RSV-specific serum antibody is detected by colorimetric detection using a secondary anti-human-IgG antibody conjugated to horse radish peroxidase (HRP) and soluble o-phenylenediamine dihydrochloride (OPD) as colorimetric substrate. The amount of bound RSV-specific serum antibody is calculated on a standard curve of purified human IgG standard, run in parallel on each assay. The RSV-specific IgG antibody titer is reported as a concentration in ng/mL serum. Full details will be outlined in the Laboratory Manual.

### RSV Neutralizing Antibody

RSV-specific serum neutralizing antibodies will be analyzed by micro neutralization assay, using RSV-A2 virus as target. Briefly, heat inactivated serum samples will be serially diluted and reacted with 100×TCID50 of RSV virus for 1h at 37° C. in tissue culture 96-wells. 20,000 Vero cells are added to each well as the virus substrate. After 72 hours of infection, assay wells are fixed and stained by immunohistochemistry with RSV mouse monoclonal antibody 2F7 (Novus Biological) and secondary goat anti-mouse-IgG conjugated to HRP and soluble OPD as colorimetric substrate. The RSV neutralization titer will be reported as the inverse of the dilution of serum that results in 50% reduction in color development [i.e., the signal of (uninhibited virus control−uninfected cell control)/2]. Full details will be outlined in the Laboratory Manual.

### Peripheral Blood Mononuclear Cell Isolation and Analysis

Blood samples for PBMC will be collected at the time points delineated in the Schedule of Assessments and will be shipped for processing and analysis as outlined the Laboratory Manual. ELISpot methods will be further outlined in a Laboratory Manual.

### Upper Airway Specimen Collection and Analysis

Nasopharyngeal swabs from both nostrils will be obtained at timepoints indicated in the Schedule of Assessments and will be processed and shipped for analysis as outlined in the Laboratory Manual. These samples will be analyzed for vaccine and RSV-specific IgA.

### Immunoglobulin A

RSV-specific IgA antibodies will be analyzed by ELISA, using RSV-A2 whole virus lysate as target antigen. Briefly, NP swabs in Universal viral transport medium (UVTM) will be serially diluted and reacted with assay wells coated with RSV antigen. Bound RSV-specific serum antibody is detected by colorimetric detection using a secondary anti-human-IgA antibody conjugated to HRP and soluble OPD as colorimetric substrate. The amount of bound

RSV-specific serum antibody is calculated on a standard curve of purified human IgA standard, run in parallel on each assay. The RSV-specific IgG antibody titer is reported as concentration in ng/mL serum. Full details will be further outlined in a Laboratory Manual.

### Testing for Symptomatic Respiratory Virus Infection

Participants with moderate to severe illness consistent with viral respiratory infection, in the opinion of the Investigator, will have NP swabs or other suitable specimens obtained for multiplex PCR panel for respiratory viruses. This can be obtained by NP swab specimen or (especially if the subject is at an outside facility) other standard specimen such as sputum or nasal wash. Any samples positive for RSV will be frozen and shipped to Codagenix for sequence analysis. Swabs will be collected, processed and shipped for analysis as outlined in the Laboratory Manual.

This testing will be done at the discretion of the investigator and may be done in addition to routine monitoring for vaccine virus shedding.

### Vaccine Shedding by PCR

Nasopharyngeal swabs will be collected. The presence and magnitude of RSV shedding in NP secretions will be assessed at the sponsor laboratory using the ARIES Flu A/B & RSV Assay (Luminex Corporation, Ref #50-10020) run on the Luminex ARIES diagnostic platform. For the purpose of quantification of RSV shedding the ARIES Flu A/B & RSV Assay is modified as a “research use only” test by calculating the amount of virus present in a sample based on a standard curve of known quantities of RSV reference virus. The shedding titer will be reported as FFU-equivalents/mL (FFUeq/mL). After each vaccination, if two sequential samples are below detection, subsequent samples will not be tested. Further details will be outlined in the Laboratory Manual.

### Sequence Analysis of Shed Virus

Isolates obtained during episodes of symptomatic RSV infection and the last positive shedding sample after each vaccination will be sequenced by next generation sequencing after first converting viral RNA to cDNA using a commercially available Reverse transcriptase PCR kit. Further details will be outlined in the Laboratory Manual.

### RSV Viremia by PCR

The presence of CodaVax-RSV in serum (viremia) will be assessed in the sponsor laboratory using the Luminex assay described in herein. Any serum samples positive by PCR will be confirmed with viral culture. Further details will be outlined in the Laboratory Manual.

### Reactogenicity

Participants will be provided with a Diary Card and thermometer after the vaccination to record oral temperature and solicited reactogenicity events and severity for 7 days following each vaccination.

Events captured on the Diary Cards will be considered Solicited Local Site Events or Solicited Systemic Events, and will be graded in line with the FDA Guidance for Industry: Toxicity Grading Scale for Healthy Adult and Adolescent Volunteers Enrolled in Preventive Vaccine Clinical Trials.

Site staff will review diaries with subjects to ensure they are legible, and that grading is consistent with toxicity grading scale provided. Site staff may ask subjects to adjust grading if indicated.

The Diary Cards will be issued to participants at Visit 2 (Day 1) and will be collected at Visit 4 (Day 8). Diary Cards will be issued again at Visit 6 (Day 29) and will be collected at Visit 8 (Day 36). If events are persisting after Day 8 or Day 36, site staff will note Study Day when symptoms ended on the source documents and enter this information into the Reactogenicity Event/Symptom CRF.

Note: Solicited reactogenicity events will not be recorded as AEs unless they are serious (i.e., they meet the criteria for an SAE).

Note: the MM will be notified if two or more participants experience similar Grade 3 events.

### Solicited Local Site Events

Local Reactogenicity symptoms collected are: Runny Nose, Nasal Congestion, Nasal Irritation/Pain, Sneezing, Coughing, Sore Throat, Eye Pain, Ear Pain, Change in Vision, Change in Smell, Change in Taste

### Solicited Systemic Events

Systemic reactogenicity symptoms collected are: Headache, Fatigue, Muscle Aches, Joint Pain, Chills, Sweating, Vomiting, Diarrhoea, Chest pain/Pleurisy, Shortness of Breath

### Fever

Body temperature (oral) should be measured by self-assessment every day and recorded in the Diary Card from each vaccination until 7 days post-vaccination. While prophylactic administration of antipyretics is not permitted within 4 hours prior to and during the first

72 hours after the vaccination, subjects may use over the counter antipyretics and/or analgesics to treat symptoms. Symptoms and temperature should be recorded at least 6 hours after last dose of such medication.

### Immunogenicity

Immunogenicity parameter values will be listed by participant and summarized by dose level (CodaVax-RSV low dose, high dose, and placebo) using the PP population. The ITT population will also be presented if more than 10% of participants excluded from PP population for any dose level. For RSV-specific IgG (in serum) and IgA (in NP swab specimens) antibodies measured by ELISA, GMT and GMR by time point will be modelled using a linear mixed effects model with participant as the random effect, timepoint (unordered) and treatment group as factors as the fixed effects and with Baseline titer as a covariate. The proportion of participants seroconverting will be tabulated as N (%) and 95% Wilson confidence intervals will be reported for each group. For neutralizing antibodies measured in serum, GMT and GMR by time point will be modelled using the same linear mixed effects model as for RSV-specific IgG above. Seroconversion rates at each timepoint and in each treatment group will be presented with 95% CIs. Scatter plots of Baseline RSV neutralizing antibody titer versus post-vaccination immunogenicity measures will be presented. T cell responses to RSV/A will be listed by participant and summarized by treatment and protocol specified time point. Subgroup analysis of immunogenicity measures by low and high Baseline RSV neutralizing antibody titer will be completed. No adjustments for multiple comparisons will be applied.

### Example 2

The following example is based on data collected in the clinical trial described by Example 1, above.

Data from the ELISpot performed by 360 Bio was reanalyzed. Detailed conditions and planned analysis are provided in the 360 Bio labs assay report and the statistical analysis plan. For this post-hoc analysis, only the values from the 1:10 stimulation for the low dose cohort (13 vaccine and 7 placebo recipients) are included.

For the post hoc analysis we evaluated change from baseline (Day 1) to the highest value at any post vaccination timepoint (Peak) and defined a responder as any subject with ≥1.3 fold increase from baseline.

Baseline and peak values were compared for the vaccine and placebo recipients using a paired T-test. Fold change from baseline to peak post vaccination timepoint was calculated by imputing values <LLOQ (50 SFU) as 49 SFU. Fold changes were compared for vaccine versus placebo group using the Mann-Whitney test.

### Results:

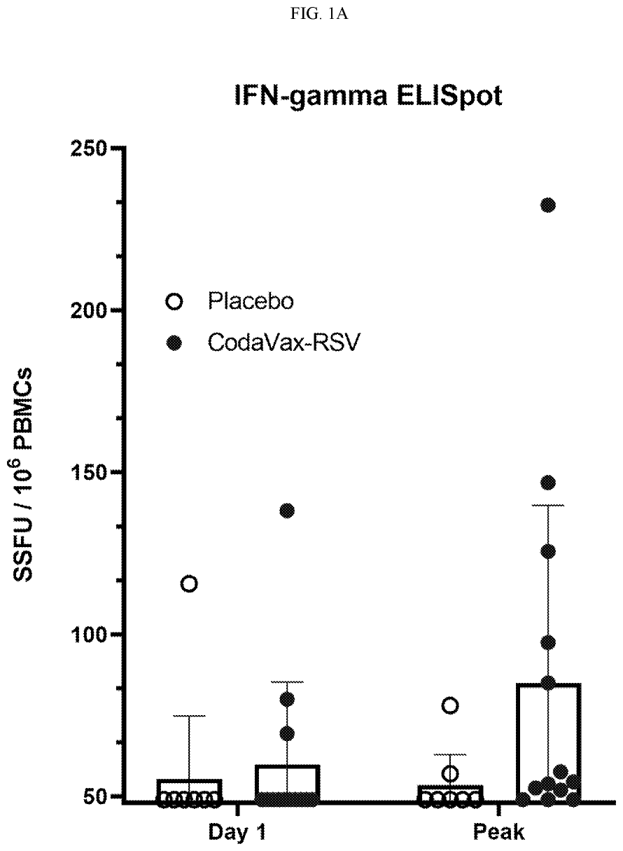

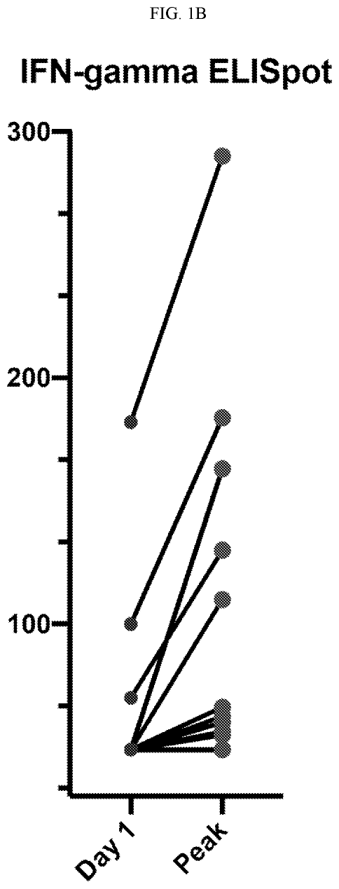

Total Detectable RSV-specific Reponses: It was shown that 10/13 of vaccinated volunteers displayed a detectable increase in the number of spot forming units compared to the day 1 indicating on the development of T cell response (76.9%). In placebo group, we saw increase only in 1/7 volunteers (14.2%). The vaccine group baseline and peak values were statistically different by the paired T-test (p<0.01) but the placebo group showed no difference (p=0.39). The values of SFU at baseline and peak for both placebo and CodaVax-RSV vaccinated volunteers are presented in FIG. 1A and the individual changes are shown in FIG. 1B.

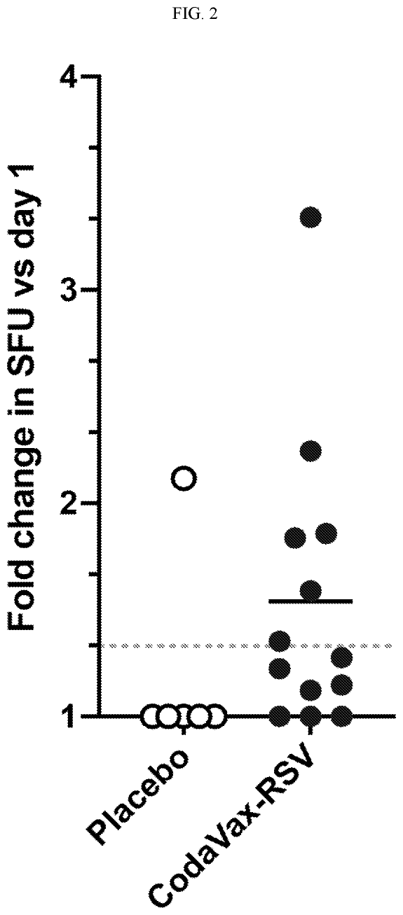

Additionally, 6/13 (46%) of CodaVax-vaccinated volunteers displayed increase in the number of SFUs greater than 1.3× compared to 1/7 (14.3%) placebo recipients. Fold change in the number of SFU on the peak day vs day 1 are illustrated in FIG. 2.

Volunteers vaccinated with CodaVax-RSV displayed on average 1.5× increase in the numbers of their SFUs compared to −0.98× fold change in the group treated with placebo. Difference in the increase of SFU between placebo and CodaVax-RSV vaccinated groups was statistically significant as evaluated with Mann-Whitney test (p<0.03).

In this post-hoc exploration of the ELISpot data a statistically significant increase in cellular immune response after vaccination was identified that was not as readily apparent in the planned analysis that was limited to evaluation of baseline to each post vaccination timepoint, rather than baseline to peak post vaccination time point. In addition, the planned analysis did not include a definition of responder as was done here.

In future studies we may use the 1.3 fold rise to define a responder and also plan to process PBMCs from whole blood within a shorter timeframe from blood draw, which may enable better detection of activated lymphocytes, which are fragile and may be lost in transport.

### Example 3

The following example is also based on data collected in the clinical trial described by Example 1, above.

RSV-specific IgA to Total IgA Antibodies Measured by ELISA in Nasopharyngeal Swabs by Visit. The results are presented below.

Summary and Analysis of the Ratio of RSV-specific IgA to Total IgA Antibodies Measured by ELISA in Nasopharyngeal Swabs by Visit and Baseline RSV Neutralizing Antibody Titer mITT Population

### Baseline Titer: <=Median

| Visit | CodaVax-RSV Dose | | | |
|---|---|---|---|---|
| Parameter | 2 × 10⁵ FFU | 2 × 10⁶ FFU | Overall | Placebo |
| Statistic | (N = 9) | (N = 2) | (N = 11) | (N = 7) |
| Day 43 | | | | |
| Subjects With a Valid Assay | 8 (88.9%) | 2 (100.0%) | 10 (90.9%) | 7 (100.0%) |
| Seroconversion Rate | 11.1% | 50.0% | 18.2% | 0% |
| 95% Exact CI | 2.0%, 43.5% | 9.5%, 90.5% | 5.1%, 47.7% | 0.0%, 35.4% |
| Geometric mean titer (GMT) | | | | |
| Mean | 0.7 | 1.2 | 0.7 | 1.0 |
| 95% CI of GMT | 0.3, 1.5 | 0.0, 111.2 | 0.4, 1.5 | 0.7, 1.4 |
| Geometric mean fold rise (GMFR) | | | | |
| Mean | 1.1 | 2.4 | 1.3 | 1.0 |
| 95% CI of GMFR | 0.7, 1.8 | 0.0, 42709.0 | 0.8, 2.2 | 0.6, 1.5 |

### Example 4

MinL-4.0 (RSV-MinL NPM2-1(N88K)L in Le Nouen et al, 2017) (SEQ ID NO:1)

ACGGGAAAAAATGCGTACAACAAACTTGCATAAACCAAAAAAATGGGGCAAATAAGAATTTGATAAGTACCACTTAAATT
TAACTCCCTTGGTTAGAGATGGGCAGCAATTCATTGAGTATGATAAAAGTTAGATTACAAAATTTGTTTGACAATGATGA
AGTAGCATTGTTAAAAATAACATGCTATACTGATAAATTAATACATTTAACTAATGCTTTGGCTAAGGCAGTGATACATA
CAATCAAATTGAATGGCATTGTGTTTGTGCATGTTATTACAAGTAGTGATATTTGCCCTAATAATAATATTGTAGTAAAA
TCCAATTTCACAACAATGCCAGTACTACAAAATGGAGGTTATATATGGGAAATGATGGAATTAACACATTGCTCTCAACC
TAATGGTCTACTAGATGACAATTGTGAAATTAAATTCTCCAAAAAACTAAGTGATTCAACAATGACCAATTATATGAATC
AATTATCTGAATTACTTGGATTTGATCTTAATCCATAAATTATAATTAATATCAACTAGCAAATCAATGTCACTAACACC
ATTAGTTAATATAAAACTTAACAGAAGACAAAAATGGGGCAAATAAATCAATTCAGCCAACCCAACCATGGACACAACCC
ACAATGATAATACACCACAAAGACTGATGATCACAGACATGAGACCGTTGTCACTTGAGACCATAATAACATCACTAACC
AGAGACATCATAACACACAAATTTATATACTTGATAAATCATGAATGCATAGTGAGAAAACTTGATGAAAGACAGGCCAC
ATTTACATTCCTGGTCAACTATGAAATGAAACTATTACACAAAGTAGGAAGCACTAAATATAAAAAATATACTGAATACA
ACACAAAATATGGCACTTTCCCTATGCCAATATTCATCAATCATGATGGGTTCTTAGAATGCATTGGCATTAAGCCTACA
AAGCATACTCCCATAATATACAAGTATGATCTCAATCCATAAATTTCAACACAATATTCACACAATCTAAAACAACAACT
CTATGCATAACTATACTCCATAGTCCAGATGGAGCCTGAAAATTATAGTAATTTAAAACTTAAGGAGAGATATAAGATAG
AAGATGGGGCAAATACAACCATGGCTCTTAGCAAAGTCAAGTTGAATGATACACTCAACAAAGATCAACTTCTGTCATCC
AGCAAATACACCATCCAACGGAGCACAGGAGATAGTATTGATACTCCTAATTATGATGTGCAGAAACACATCAATAAGTT
ATGTGGCATGTTATTAATCACAGAAGATGCTAATCATAAATTCACTGGGTTAATAGGTATGTTATATGCGATGTCTAGGT
TAGGAAGAGAAGACACCATAAAAATACTCAGAGATGCGGGATATCATGTAAAAGCAAATGGAGTAGATGTAACAACACAT
CGTCAAGACATTAATGGAAAAGAAATGAAATTTGAAGTGTTAACATTGGCAAGCTTAACAACTGAAATTCAAATCAACAT
TGAGATAGAATCTAGAAAATCCTACAgAAAAATGCTAAAAGAAATGGGAGAGGTAGCTCCAGAATACAGGCATGACTCTC
CTGATTGTGGGATGATAATATTATGTATAGCAGCATTAGTAATAACTAAATTAGCAGCAGGGGACAGATCTGGTCTTACA
GCCGTGATTAGGAGAGCTAATAATGTCCTAAAAAATGAAATGAAACGTTACAAAGGCTTACTACCCAAGGACATAGCCAA
CAGCTTCTATGAAGTGTTTGAAAAACATCCCCACTTTATAGATGTTTTTGTTCATTTTGGTATAGCACAATCTTCTACCA
GAGGTGGCAGTAGAGTTGAAGGGATTTTTGCAGGATTGTTTATGAATGCCTATGGTGCAGGGCAAGTGATGTTACGGTGG
GGAGTCTTAGCAAAATCGGTTAAAAATATTATGTTAGGACATGCTAGTGTGCAAGCAGAAATGGAACAAGTTGTTGAGGT
TTATGAATATGCCCAAAAATTGGGTGGTGAAGCAGGATTCTACCATATATTGAACAACCCAAAAGCATCATTATTATCTT
TGACTCAATTTCCTCACTTCTCCAGTGTAGTATTAGGCAATGCTGCTGGCCTAGGCATAATGGGAGAGTACAGAGGTACA
CCGAGGAATCAAGATCTATATGATGCAGCAAAGGCATATGCTGAACAACTCAAAGAAAATGGTGTGATTAACTACAGTGT
ACTAGACTTGACAGCAGAAGAACTAGAGGCTATCAAACATCAGCTTAATCCAAAAGATAATGATGTAGAGCTTTGAGTTA
ATAAAAAATGGGGCAAATAAATCATCATGGAAAAGTTTGCTCCTGAATTCCATGGAGAAGATGCAAACAACAGGGCTACT
AAATTCCTAGAATCAATAAAGGGCAAATTCACATCACCCAAAGATCCCAAGAAAAAAGATAGTATCATATCTGTCAACTC
AATAGATATAGAAGTAACCAAAGAAAGCCCTATAACATCAAATTCAACTATTATCAACCCAACAAATGAGACAGATGATA
CTGCAGGGAACAAGCCCAATTATCAAAGAAAACCTCTAGTAAGTTTCAAAGAAGACCCTACACCAAGTGATAATCCCTTT
TCTAAACTATACAAAGAAACCATAGAAACATTTGATAACAATGAAGtAGAATCCAGCTATTCATACGAAGAAATAAATGA
TCAGACAAACGATAATATAACAGCAAGATTAGATAGGATTGATGAAAAATTAAGTGAAATACTAGGAATGCTTCACACAT
TAGTAGTGGCAAGTGCAGGACCTACATCTGCTCGGGATGGTATAAGAGATGCCATGGTTGGTTTAAGAGAAGAAATGATA
GAAAAAATCAGAACTGAAGCATTAATGACCAATGACAGATTAGAAGCTATGGCAAGACTCAGGAATGAGGAAAGTGAAAA
GATGGCAAAAGACACATCAGATGAAGTGTCTCTCAATCCAACATCAGAGAAATTGAACAACCTATTGGAAGGGAATGATA
GTGACAATGATCTATCACTTGAAGATTTCTGATTAGTTACCAATCTTCACATCAACACACAATACCAACAGAAGACCAAC
AAACTAACCAACCCAATCATCCAACCAAACATCCATCCGCCAATCAGCCAAACAGCCAACAAAACAACCAGCCAATCCAA
AACTAACCACCCGGAAAAAATCTATAATATAGTTACAAAAAAAGGAAAGGGTGGGGCAAATATGGAAACATACGTGAACA
AGCTTCACGAAGGCTCCACATACACAGCTGCTGTTCAATACAATGTCTTAGAAAAAGACGATGACCCTGCATCACTTACA
ATATGGGTGCCCATGTTCCAATCATCTATGCCAGCAGATTTACTTATAAAAGAACTAGCTAATGTCAACATACTAGTGAA
ACAAATATCCACACCCAAGGGACCTTCACTAAGAGTCATGATAAACTCAAGAAGTGCAGTGCTAGCACAAATGCCCAGCA
AATTTACCATATGCGCTAATGTGTCCTTGGATGAAAGAAGCAAACTAGCATATGATGTAACCACACCCTGTGAAATCAAG
GCATGTAGTCTAACATGCCTAAAATCAAAAAATATGTTGACTACAGTTAAAGATCTCACTATGAAGACACTCAACCCTAC
ACATGATATTATTGCTTTATGTGAATTTGAAAACATAGTAACATCAAAAAAAGTCATAATACCAACATACCTAAGATCCA
TCAGTGTCAGAAATAAAGATCTGAACACACTTGAAAATATAACAACCACTGAATTCAAAAATGCTATCACAAATGCAAAA
ATCATCCCTTACTCAGGATTACTATTAGTCATCACAGTGACTGACAACAAAGGAGCATTCAAATACATAAAGCCACAAAG
TCAATTCATAGTAGATCTTGGAGCTTACCTAGAAAAAGAAAGTATATATTATGTTACCACAAATTGGAAGCACACAGCTA
CACGATTTGCAATCAAACCCATGGAAGATTAACCTTTTTCCTCTACATCAGTGTGTTAATTCATACAAACTTTCTACCTA
CATTCTTCACTTCACCATCACAATCACAAACACTCTGTGGTTCAACCAATCAAACAAAACTTATCTGAAGTCCCAGATCA
TCCCAAGTCATTGTTTATCAGATCTAGTACTCAAATAAGTTAATAAAAAATATACACATGGGGCAAATAATCATTGGAGG
AAATCCAACTAATCACAATATCTGTTAACATAGACAAGTCCACACACCATACAGAATCAACCAATGGAAAATACATCCAT
AACAATAGAATTCTCAAGCAAATTCTGGCCTTACTTTACACTAATACACATGATCACAACAATAATCTCTTTGCTAATCA
TAATCTCCATCATGATTGCAATACTAAACAAACTTTGTGAATATAACGTATTCCATAACAAAACCTTTGAGTTACCAAGA
GCTCGAGTTAATACTTGATAAAGTAGTTAATTAAAAATAGTCATAACAATGAACTAGGATATCAAGACTAACAATAACAT
TGGGGCAAATGCAAACATGTCCAAAAACAAGGACCAACGCACCGCTAAGACATTAGAAAGGACCTGGGACACTCTCAATC
ATTTATTATTCATATCATCGTGCTTATATAAGTTAAATCTTAAATCTGTAGCACAAATCACATTATCCATTCTGGCAATG
ATAATCTCAACTTCACTTATAATTGCAGCCATCATATTCATAGCCTCGGCAAACCACAAAGTCACACCAACAACTGCAAT
CATACAAGATGCAACAAGCCAGATCAAGAACACAACCCCAACATACCTCACCCAGAATCCTCAGCTTGGAATCAGTCCCT
CTAATCCGTCTGAAATTACATCACAAATCACCACCATACTAGCTTCAACAACACCAGGAGTCAAGTCAACCCTGCAATCC
ACAACAGTCAAGACCAAAAACACAACAACAACTCAAACACAACCCAGCAAGCCCACCACAAAACAACGCCAAAACAAACC
ACCAAGCAAACCCAATAATGATTTTCACTTTGAAGTGTTCAACTTTGTACCCTGCAGCATATGCAGCAACAATCCAACCT
GCTGGGCTATCTGCAAAAGAATACCAAACAAAAAACCAGGAAAGAAAACCACTACCAAGCCCACAAAAAAACCAACCCTC
AAGACAACCAAAAAAGATCCCAAACCTCAAACCACTAAATCAAAGGAAGTACCCACCACCAAGCCCACAGAAGAGCCAAC
CATCAACACCACCAAAACAAACATCATAACTACACTACTCACCTCCAACACCACAGGAAATCCAGAACTCACAAGTCAAA
TGGAAACCTTCCACTCAACTTCCTCCGAAGGCAATCCAAGCCCTTCTCAAGTCTCTACAACATCCGAGTACCCATCACAA
CCTTCATCTCCACCCAACACACCACGCCAGTAGTTACTTAAAAACATATTATCACAAAAGGCCTTGACCAACTTAAACAG
AATCAAAATAAACTCTGGGGCAAATAACAATGGAGTTGCTAATCCTCAAAGCAAATGCAATTACCACAATCCTCACTGCA
GTCACATTTTGTTTTGCTTCTGGTCAAAACATCACTGAAGAATTTTATCAATCAACATGCAGTGCAGTTAGCAAAGGCTA
TCTTAGTGCTCTGAGAACTGGTTGGTATACCAGTGTTATAACTATAGAATTAAGTAATATCAAGAAAAATAAGTGTAATG
GAACAGATGCTAAGGTAAAATTGATAAAACAAGAATTAGATAAATATAAAAATGCTGTAACAGAATTGCAGTTGCTCATG
CAAAGCACACAAGCAACAAACAATCGAGCCAGAAGAGAACTACCAAGGTTTATGAATTATACACTCAACAATGCCAAAAA
AACCAATGTAACATTAAGCAAGAAAAGGAAAAGAAGATTTCTTGGTTTTTTGTTAGGTGTTGGATCTGCAATCGCCAGTG
GCGTTGCTGTATCTAAGGTCCTGCACCTAGAAGGGGAAGTGAACAAGATCAAAAGTGCTCTACTATCCACAAACAAGGCT
GTAGTCAGCTTATCAAATGGAGTTAGTGTTTTAACCAGCAAAGTGTTAGACCTCAAAAACTATATAGATAAACAATTGTT
ACCTATTGTGAACAAGCAAAGCTGCAGCATATCAAATATAGAAACTGTGATAGAGTTCCAACAAAAGAACAACAGACTAC
TAGAGATTACCAGGGAATTTAGTGTTAATGCAGGCGTAACTACACCTGTAAGCACTTACATGTTAACTAATAGTGAATTA
TTGTCATTAATCAATGATATGCCTATAACAAATGATCAGAAAAAGTTAATGTCCAACAATGTTCAAATAGTTAGACAGCA
AAGTTACTCTATCATGTCCATAATAAAAGAGGAAGTCTTAGCATATGTAGTACAATTACCACTATATGGTGTTATAGATA
CACCCTGTTGGAAACTACACACATCCCCTCTATGTACAACCAACACAAAAGAAGGGTCCAACATCTGTTTAACAAGAACT
GACAGAGGATGGTACTGTGACAATGCAGGATCAGTATCTTTCTTCCCACAAGCTGAAACATGTAAAGTTCAATCAAATCG
AGTATTTTGTGACACAATGAACAGTTTAACATTACCAAGTGAAGTAAATCTCTGCAATGTTGACATATTCAACCCCAAAT
ATGATTGTAAAATTATGACTTCAAAAACAGATGTAAGCAGCTCCGTTATCACATCTCTAGGAGCCATTGTGTCATGCTAT
GGCAAAACTAAATGTACAGCATCCAATAAAAATCGTGGAATCATAAAGACATTTTCTAACGGGTGCGATTATGTATCAAA
TAAAGGGGTGGACACTGTGTCTGTAGGTAACACATTATATTATGTAAATAAGCAAGAAGGTAAAAGTCTCTATGTAAAAG
GTGAACCAATAATAAATTTCTATGACCCATTAGTATTCCCCTCTGATGAATTTGATGCATCAATATCTCAAGTCAACGAG
AAGATTAACCAGAGCCTAGCATTTATTCGTAAATCCGATGAATTATTACATAATGTAAATGCTGGTAAATCCACCACAAA
TATCATGATAACTACTATAATTATAGTGATTATAGTAATATTGTTATCATTAATTGCTGTTGGACTGCTCTTATACTGTA
AGGCCAGAAGCACACCAGTCACACTAAGCAAAGATCAACTGAGTGGTATAAATAATATTGCATTTAGTAACTAAATAAAA
ATAGCACCTAATCATGTTCTTACAATGGTTTACTATCTGCTCATAGACAACCCATCTGTCATTGGATTTTCTTAAAATCT
GAACTTCATCGAAACTCTCATCTATAAACCATCTCACTTACACTATTTAAGTAGATTCCTAGTTTATAGTTATATAAAAC
ACAATTGCATGCCAGATTAACTTACCATCTGTAAAAATGAAAACTGGGGCAAATATGTCACGAAGGAATCCTTGCAAATT
TGAAATTCGAGGTCATTGCTTAAATGGTAAGAGGTGTCATTTTAGTCATAATTATTTTGAATGGCCACCCCATGCACTGC
TTGTAAGACAAAACTTTATGTTAAACAGAATACTTAAGTCTATGGATAAAAGTATAGATACCTTATCAGAAATAAGTGGA
GCTGCAGAGTTGGACAGAACAGAAGAGTATGCTCTTGGTGTAGTTGGAGTGCTAGAGAGTTATATAGGATCAATAAAaAA
TATAACTAAACAATCAGCATGTGTTGCCATGAGCAAACTCCTCACTGAACTCAATAGTGATGATATCAAAAAGCTGAGGG
ACAATGAAGAGCTAAATTCACCCAAGATAAGAGTGTACAATACTGTCATATCATATATTGAAAGCAACAGGAAAAACAAT
AAACAAACTATCCATCTGTTAAAAAGATTGCCAGCAGACGTATTGAAGAAAACCATCAAAAACACATTGGATATCCATAA
GAGCATAACCATCAACAACCCAAAAGAATCAACTGTTAGTGATACAAATGACCATGCCAAAAATAATGATACTACCTGAC
AAATATCCTTGTAGTATAACTTCCATACTAATAACAAGTAGATGTAGAGTTACTATGTATAATCAAAAGAACACACTATA
TTTCAATCAAAACAACCCAAATAACCATATGTACTCACCGAATCAAACATTCAATGAAATCCATTGGACCTCTCAAGAAT
TGATTGACACAATTCAAAATTTTCTACAACATCTAGGTATTATTGAGGATATATATACAATATATATATTAGTGTCATAA
CACTCAATTCTAACACTCACCACATCGTTACATTATTAATTCAAACAATTCAAGTTGTGGGACAAAATGGATCCCATTAT
TAATGGAAATTCTGCTAACGTATACTTAACCGATAGTTATTTAAAAGGCGTAATCAGTTTTAGCGAATGTAACGCATTAG
GGTCATATATCTTTAACGGTCCATATCTTAAAAACGATTATACTAATCTAATCAGTAGACAGAATCCGTTAATCGAACAT
ATGAATCTTAAgAAACTGAATATCACACAATCTTTGATCAGTAAGTATCATAAAGGCGAAATCAAACTCGAAGAACCTAC
ATATTTTCAATCACTATTAATGACATATAAGTCTATGACATCTAGCGAACAGATCGCTACTACTAATCTGTTgAAgAAAA
TTATTAGACGAGCTATAGAGATATCTGACGTTAAGGTATACGCTATACTGAATAAaTTgGGGTTAAAAGAGAAAGATAAG
ATAAAATCTAATAACGGTCAAGACGAAGATAATAGTGTAATTACTACAATTATTAAAGACGATATACTATCCGCAGTgAA
GGATAATCAATCACATCTTAAAGCCGATAAAAATCATAGTACTAAACAAAAAGATACAATTAAAACTACATTGTTAAAgA
AATTGATGTGTTCTATGCAACATCCACCTAGTTGGTTAATACATTGGTTTAACTTATACACTAAGTTgAACAATATACTT
ACACAATATCGATCAAACGAAGTgAAAAATCACGGTTTTACATTGATAGATAATCAAACATTAAGCGGATTTCAATTCAT
ACTTAACCAATACGGATGTATAGTGTATCATAAAGAaTTgAAACGTATAACCGTTACAACATATAATCAATTCTTAACAT
GGAAAGATATAAGTCTATCTAGATTGAACGTATGCTTAATTACATGGATTTCGAATTGTCTTAATACACTTAATAAATCA
TTAGGGTTAAGATGCGGATTTAATAACGTTATACTTACACAATTGTTCTTATACGGAGATTGTATACTTAAGTTgTTCCA
TAACGAAGGGTTTTATATAATAAAAGAGGTTGAGGGATTTATAATGTCATTGATACTGAATATTACCGAAGAGGATCAAT
TTAGAAAAAGATTCTATAATAGTATGTTAAACAATATAACTGACGCAGCTAATAAAGCGCAgAAgAATCTGTTATCTAGA
GTATGTCATACATTGTTAGACAAAACAGTgAGCGATAATATTATAAACGGTAGATGGATTATACTGTTATCTAAATTCTT
AAAATTGATTAAGTTgGCAGGTGACAATAACCTTAATAACTTAAGCGAATTGTATTTCTTATTCAGAATATTCGGACATC
CTATGGTTGACGAACGACAAGCTATGGACGCAGTgAAGATTAATTGTAACGAAACTAAATTCTATCTATTATCTAGTCTA
TCTATGCTTAGAGGCGCATTCATATATAGAATTATAAAAGGGTTCGTTAATAATTATAATAGATGGCCTACACTTAGAAA
CGCTATAGTGTTACCACTTAGATGGTTAACATATTATAAATTGAATACATATCCTAGTTTACTCGAATTAACCGAACGCG
ATCTGATAGTGTTAAGCGGACTTAGATTCTATAGAGAGTTTAGATTGCCTAAgAAAGTCGATCTCGAAATGATAATTAAC
GATAAGGCAATTAGTCCACCTAAAAACTTAATATGGACAAGcTTCCCTAGAAATTATATGCCTAGTCATATACAAAATTA
TATCGAACACGAAAAATTGAAATTTAGCGAATCCGATAAGTCTAGAAGAGTGTTAGAGTATTACTTACGCGATAATAAAT
TTAACGAATGCGATCTATATAATTGCGTAGTgAACCAATCATATCTTAATAATCCTAATCACGTAGTgAGTCTTACAGGT
AAGGAAAGAGAGTTgAGCGTAGGTAGAATGTTCGCTATGCAACCCGGTATGTTTAGACAAGTGCAAATACTCGCAGAAAA
gATGATAGCCGAAAATATACTGCAATTcTTTCCCGAATCATTGACTAGATACGGAGATTTAGAaTTgCAAAAgATACTCG
AATTGAAAGCAGGTATATCTAATAAGTCTAATAGATATAACGATAATTATAATAATTATATATCTAAGTGTAGTATTATT
ACCGATCTATCTAAATTCAATCAGGCATTTAGATACGAAACTAGTTGTATATGCTCAGACGTATTAGACGAATTACACGG
AGTGCAATCTTTGTTTAGTTGGTTACATTTAACTATACCTCACGTTACAATTATATGTACATATAGACACGCACCACCAT
ATATAGGCGATCATATAGTCGATCTGAATAACGTAGACGAACAATCCGGATTGTATAGATATCACATGGGTGGCATAGAG
GGATGGTGTCAAAAATTGTGGACTATAGAGGCAATTAGTCTGTTAGATCTAATTAGTCTTAAGGGTAAGTTTTCGATTAC
CGCATTGATTAACGGTGATAATCAATCAATTGATATATCTAAACCGATACGGTTAATGGAGGGACAAACACACGCTCAAG
CCGATTACTTACTCGCACTTAATTCACTTAAACTGTTATACAAAGAGTACGCAGGTATAGGGCATAAACTTAAGGGTACA
GAGACATATATAAGTAGGGATATGCAATTTATGAGTAAGACTATACAACATAACGGAGTGTATTATCCCGCTAGTATAAA
gAAAGTGCTTAGAGTCGGACCTTGGATTAATACTATATTAGACGATTTTAAGGTTAGTCTCGAATCAATCGGATCATTGA
CACAAGAGTTgGAGTATAGAGGCGAATCTCTATTATGCTCATTGATTTTTAGAAACGTATGGTTATACAATCAGATTGCA
TTGCAATTGAAAAATCACGCACTATGTAATAATAAGTTgTACTTAGACATACTTAAAGTGTTAAAACATCTTAAAACATT
cTTTAATCTCGATAATATAGATACCGCATTAACATTGTATATGAATCTACCTATGTTATTCGGAGGGGGAGATCCTAATC
TATTGTATAGATCATTCTATAGACGTACACCTGATTTCTTAACCGAAGCTATAGTGCATAGCGTATTCATACTATCATAT
TATACTAATCACGATCTTAAAGATAAGTTgCAGGATCTATCTGACGATAGATTGAATAAATTCTTAACATGTATTATAAC
ATTCGATAAAAATCCTAACGCTGAATTCGTTACACTTATGAGAGATCCACAAGCATTAGGTTCAGAGAGACAGGCTAAAA
TTACTAGCGAAATTAATAGATTAGCCGTTACCGAAGTGTTAAGTACCGCACCTAATAAGATATTCTCTAAATCCGCTCAA
CATTATACAACAACCGAAATAGATCTTAACGATATTATGCAAAATATCGAACCTACATATCCTCACGGATTACGCGTAGT
TTACGAATCATTACCATTCTATAAAGCCGAAAAgATCGTTAACTTAATTAGCGGTACAAAATCAATTACTAATATACTCG
AAAAgACTAGCGCAATTGATTTAACCGATATAGATAGAGCTAtCGAAATGATGCGTAAAAATATAACATTACTGATACGT
ATACTACCATTAGATTGTAATAGGGATAAAAGAGAGATACTATCTATGGAGAATCTATCAATTACAGAaTTgTCAAAATA
CGTTAGGGAACGATCATGGTCACTATCTAATATCGTAGGCGTAACTAGTCCTAGTATTATGTATACTATGGATATTAAGT
ATACAACTAGTACAATTAGTAGCGGTATAATAATCGAAAAATATAACGTTAATAGTCTAACACGTGGTGAAAGGGGACCT
ACAAAACCTTGGGTCGGATCTAGTACACAAGAGAAgAAAACTATGCCCGTATATAATAGACAGGTATTGACTAAgAAACA
ACGAGATCAAATAGATCTATTAGCTAAACTCGATTGGGTATACGCTAGTATAGATAATAAAGACGAATTTATGGAAGAGT
TgTCAATCGGTACATTAGGGTTAACATACGAAAAAGCTAAgAAATTGTTCCCACAATATCTATCAGTgAATTATCTACAT
AGATTGACAGTgAGTAGTAGACCATGCGAATTTCCCGCTAGTATACCCGCATATAGAACTACTAATTATCATTTCGATAC
TAGTCCAATTAATAGAATATTAACCGAAAAATACGGAGACGAAGATATAGATATCGTATTCCAAAATTGTATTAGTTTCG
GATTGAGTCTTATGTCCGTAGTCGAACAATTTACTAACGTATGTCCTAATAGGATTATACTGATACCTAAaTTgAACGAA
ATACATCTTATGAAACCTCCTATTTTTACAGGCGATGTCGATATACAcAAATTGAAACAGGTTATACAAAAACAACATAT
GTTCTTACCCGATAAGATATCGTTAACGCAATACGTTGAGTTgTTCTTATCAAATAAAACACTTAAATCAGGTAGTCACG
TTAATAGTAATCTGATACTCGCACATAAAATTAGCGATTACTTTCATAATACATATATATTGAGTACTAACTTAGCCGGA
CATTGGATACTGATTATACAATTGATGAAAGATAGTAAGGGTATATTCGAAAAAGATTGGGGTGAGGGATATATAACCGA
TCATATGTTTATAAACCTTAAGGTcTTcTTTAACGCATATAAAACTTATCTATTATGTTTTCATAAGGGATACGGTAAGG
CTAAACTCGAATGCGATATGAATACATCCGATCTATTATGCGTACTCGAATTAATTGATAGTAGcTATTGGAAATCTATG
AGTAAGGTATTCTTAGAGCAAAAgGTGATcAAgTATATACTATCTCAAGACGCTAGTTTGCATAGGGTTAAGGGATGTCA
TAGTTTTAAaTTATGGTTTCTTAAAAGATTGAACGTAGCCGAATTTACAGTATGTCCTTGGGTCGTTAACATAGATTATC
ATCCTACACATATGAAAGCTATACTTACATATATAGATCTAGTgAGAATGGGATTGATTAACATAGATAGAATACATATA
AAgAATAAACATAAATTTAACGACGAATTCTATACTAGTAATCTATTCTATATAAATTATAATTTTTCCGATAATACACA
TCTATTAACTAAACATATACGTATAGCTAATAGCGAACTCGAAAATAATTATAATAAATTGTATCATCCTACACCCGAAA
CATTAGAGAATATACTCGCTAATCCGATTAAATCTAACGATAAgAAAACACTTAACGATTATTGTATAGGTAAAAACGTT
GATTCAATTATGTTACCATTACTATCAAATAAgAAATTGATTAAATCTAGCGCTATGATTAGAACTAATTATAGTAAACA
GGATCTATATAACTTATTCCCTATGGTCGTAATTGATAGAATTATAGATCATTCCGGTAATACCGCTAAATCTAATCAAT
TGTATACAACTACTAGTCATCAAATATCATTAGTGCATAATAGTACTAGTCTATATTGTATGTTACCATGGCATCATATT
AATAGATTCAATTTCGTTTTTAGTAGTACAGGGTGTAAAATTAGTATAGAGTATATACTTAAAGATCTTAAAATTAAAGA
TCCTAATTGTATTGCATTCATAGGCGAAGGCGCAGGTAATCTGTTACTTAGAACAGTAGTCGAATTGCATCCCGATATTA
GATATATATATAGATCACTTAAAGATTGTAACGATCATAGTCTACCAATCGAATTCCTTAGATTGTATAACGGTCATATA
AACATAGATTACGGCGAAAACTTAACGATACCCGCTACTGACGCTACTAATAATATACATTGGTCATACTTACATATTAA
ATTCGCAGAACCTATAAGTCTATTCGTATGCGACGCAGAaTTATCCGTTACAGTgAATTGGTCTAAAATTATTATCGAAT
GGTCTAAACACGTTAGAAAATGCAAATATTGTTCTAGCGTTAATAAGTGTATGTTAATCGTTAAGTATCACGCTCAAGAC
GATATAGATTTTAAaTTAGATAATATAACTATACTTAAAACATACGTATGCTTAGGTAGTAAGCTTAAGGGTAGCGAAGT
ATACTTAGTGTTAACGATAGGTCCAGCTAATATTTTTCCCGTTTTTAACGTAGTGCAAAACGCTAAATTGATTCTATCTA
GAACTAAAAATTTtATAATGCCTAAgAAAGCTGATAAAGAGTCAATTGACGCTAATATAAAATCATTGATACCATTCTTA
TGTTATCCTATAACTAAgAAAGGGATTAATACCGCACTATCTAAACTTAAATCCGTAGTgAGCGGAGATATACTATCTTA
TAGTATAGCCGGTAGAAACGAAGTTTTTAGTAATAAATTGATTAATCATAAACATATGAATATACTTAAATGGTTTAATC
ACGTACTTAATTTTAGATCAACCGAATTGAATTATAATCATCTATATATGGTCGAATCTACATATCCATACTTATCCGAA
CTGTTAAACTCATTGACTACTAACGAATTGAAgAAATTGATTAAAATTACAGGTAGTCTGTTATACAATTTTCATAACGA
ATAATGAATAAAGATCTTATAATAAAAATTCCCATAGCTATACACTAACACTGTATTCAATTATAGTTATTAAAAATTAA
AAATCATATAATTTTTTAAATAACTTTTAGTGAACTAATCCTAAAGTTATCATTTTAATCTTGGAGGAATAAATTTAAAC
CCTAATCTAATTGGTTTATATGTGTATTAACTAAATTACGAGATATTAGTTTTTGACACTTTTTTTCTCGT

Four nucleotide difference compared to MinL parent (introduced by reverse genetics, see Le Nouen 2017) in RSV-MinL NPM2-1(N88K)L (MinL4.0) are: A1547G, A2687T, C7758A, C11883T.

```
FEATURES Location/Qualifiers
  misc_feature 1..15111
    /note=“RSV D46_6120”
  CDS 99..518
    /note=“NS1”
  CDS 628..1002
    /note=“NS2”
  CDS 1141..2316
    /note=“N”
  CDS 2347..3072
    /note=“P”
  CDS 3262..4032
    /note=“M”
  CDS 3971..4198
    /note=“Putative alternate translation of M”
  CDS 4304..4498
    /note=“SH”
  CDS 4577..5473
    /note=“G”
  CDS 5550..7274
    /note=“F”
  CDS 7495..8079
    /note=“M2-1”
  CDS 8048..8320
    /note=“M2-2”
  CDS 8387..14884
    /note=“L”
  misc_feature 45..53
    /note=“NS1 Gene Start”
  misc_feature 596..604
    /note=“NS2 gene start”
  misc_feature 1126..1134
    /note=“N Gene Start”
  misc_feature 2330..2338
    /note=“P Gene Start”
  misc_feature 3253..3261
    /note=“M Gene Start”
  misc_feature 4220..4228
    /note=“SH Gene Start”
  misc_feature 4562..4570
    /note=“G Gene Start”
  misc_feature 5537..5545
    /note=“F Gene Start”
  misc_feature 7486..7494
    /note=“M2 Gene Start”
  misc_feature 8379..8386
    /note=“L Gene Start”
  misc_feature 14945..14956
    /note=“L Gene End”
  misc_feature 564..576
    /note=“NS1 Gene End”
  misc_feature 1087..1098
    /note=“NS2 Gene End”
  misc_feature 2316..2328
    /note=“N Gene End”
  misc_feature 4198..4210
    /note=“M Gene End”
  misc_feature 3231..3243
    /note=“P Gene End”
  misc_feature 4505..4517
    /note=“SH Gene End”
  misc_feature 5472..5484
    /note=“G Gene End”
  misc_feature 7428..7439
    /note=“F Gene End”
  misc_feature 8435..8446
    /note=“M2 Gene End”
```

### Example 5

### T-Cell Response

The following example is also based on data collected in the clinical trial described by Example 1, above.

No difference between CodaVax-RSV dose groups or overall CodaVax-RSV group and the placebo group was seen in planned analysis of postdose T-cell response measured by RSV-stimulated PBMCs (geometric mean SFU) in either analysis population. GMFRs ranged from 0.9 to 1.3.

In the post hoc analysis defining a responder as a participant with any increase from baseline to peak value, the overall CodaVax-RSV group had a responder rate of 66.7% (95% CI: 46.7, 82.0%), compared to placebo 16.7% (95% CI: 4.7, 44.8%).

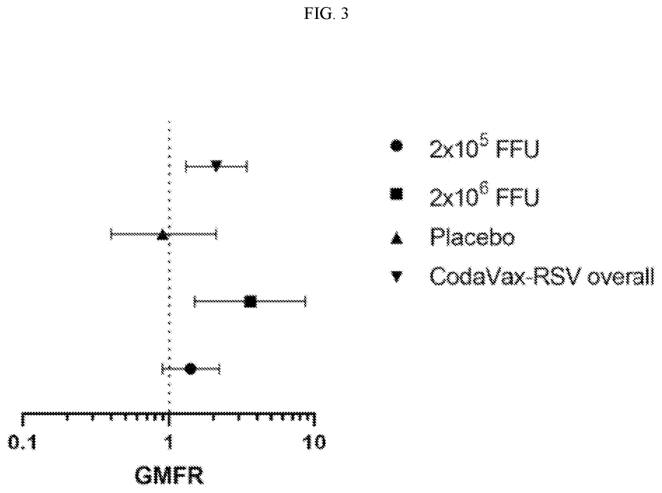

The analysis was repeated with the raw data (rather than imputing values less than LLOQ as the LLOQ, which masked changes in SFU<50), In this analysis, the participants who received Coda Vax-RSV had increases in SFU, but no change was seen in placebo recipients. A potential dose effect can be seen in this response, as shown in FIG. 3. The Day 8 GMFR using the 1/10 viral lysate stimulus was 3.6 (95% CI: 1.5, 8.7) for the 2×10⁶ PFU group, 1.4 (95% CI: 0.9, 2.2) for the 2×10⁵ PFU group, and 2.1 (95% CI: 1.3, 3.4) for the overall Coda Vax-RSV groups, while the placebo group did not change (GMFR: 0.9 [95% CI: 0.4, 2.1]). Differences were similar at the other timepoints and with the 1/80 viral lysate (FIG. 3).

The cellular immune responses appeared after Dose 1.

In FIG. 3, the vertical dotted line (1-fold change) means no change. The lines around the point estimate indicate the 95% confidence interval. Thus, the fact that the confidence intervals for high dose and active group overall do not cross 1 shows that there is a meaningful increase in the T-cell response. The placebo group does not change and the confidence interval goes both sides of that dotted line. For the low dose group, there was a trend to an increase, but the fact that the confidence interval crossed below 1 indicates that it is not statistically significant.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of eliciting an immune response in a subject, comprising
intranasally administering one or more doses of an effective amount of a composition comprising a deoptimized respiratory syncytial virus (RSV) to the subject to elicit the immune response,
wherein the effective amount is about 10³-10⁹ focus forming units (FFU) of the deoptimized RSV,
wherein the immune response comprises IgA immune response, cellular immune response or both.

2. The method of claim 1, wherein intranasally administering one or more doses of the effective amount of the composition comprises administering two doses, wherein a second dose is administered about 28 days after a first dose.

3. The method of claim 1, wherein intranasally administering one or more doses of the effective amount of the composition comprises administering two doses, wherein a second dose is administered at least 28 days after a first dose.

4. The method of claim 1, wherein intranasally administering one or more doses of the effective amount of the composition comprises administering two doses, wherein a second dose is administered about 28-35 days after a first dose.

5. The method of claim 1, wherein intranasally administering comprises administering via nose drops or via nasal spray.

6. (canceled)

7. The method claim 1, wherein the effective amount is about 10⁵, about 2×10⁵, about 10⁶, or about 2×10⁶.

8. (canceled)

9. (canceled)

10. (canceled)

11. The method of claim 1, wherein one dose of the effective amount is provided in a volume of about 1 mL.

12. The method of claim 1, wherein one dose of the effective amount is administered in about four aliquots.

13. The method of claim 12,
wherein a first aliquot is administered in a first nostril, and a second aliquot is administered in a second nostril within 10 minutes of the first aliquot being administered in the first nostril, and
wherein a third aliquot and a fourth aliquot are administered in to the first nostril and the second nostril, in either order, about 5-20 minutes after the second aliquot is administered into the second nostril.

14. The method of claim 12,
wherein a first aliquot is administered in a first nostril, and a second aliquot is administered in a second nostril within 5 minutes of the first aliquot being administered in the first nostril, and
wherein a third aliquot and a fourth aliquot are administered in to the first nostril and the second nostril, in either order, about 10-15 minutes after the second aliquot is administered into the second nostril.

15. The method of claim 1, wherein the cellular immune response comprises a development of a T-cell response.

16. The method of claim 1, wherein the development of the T-cell response is in comparison to a reference level of response based on a placebo group or in comparison to a prevaccination timepoint for the subject.

17. The method of claim 1,
wherein an enzyme-linked immune absorbent spot (ELISpot) analysis of peripheral blood mononuclear cells (PBMCs) from the subject shows at least a 1.3 fold increase of spot forming units (SFU) between a baseline value and a highest value during a post vaccination timepoint, or
wherein an enzyme-linked immune absorbent spot (ELISpot) analysis of peripheral blood mononuclear cells (PBMCs) from the subject shows at least a 1.4 fold increase of spot forming units (SFU) between a baseline value and a highest value during a post vaccination timepoint, or
wherein an enzyme-linked immune absorbent spot (ELISpot) analysis of peripheral blood mononuclear cells (PBMCs) from the subject shows at least a 1.5 fold increase of spot forming units (SFU) between a baseline value and a highest value during a post vaccination timepoint, or
wherein an enzyme-linked immune absorbent spot (ELISpot) analysis of peripheral blood mononuclear cells (PBMCs) from the subject shows at least a 1.6 fold increase of spot forming units (SFU) between a baseline value and a highest value during a post vaccination timepoint, or
wherein an enzyme-linked immune absorbent spot (ELISpot) analysis of peripheral blood mononuclear cells (PBMCs) from the subject shows at least a 1.7 fold increase of spot forming units (SFU) between a baseline value and a highest value during a post vaccination timepoint.

18. (canceled)

19. (canceled)

20. (canceled)

21. (canceled)

22. The method of claim 1,
wherein the effective amount is about 10⁶ FFU and eliciting the immune response comprises a statistically significant increase in T-cell response as compared to a prevaccination timepoint, or
wherein the effective amount is about 2×10⁶ FFU and eliciting the immune response comprises a statistically significant increase in T-cell response as compared to a prevaccination timepoint, or
wherein the effective amount is about 10⁵ FFU and eliciting the immune response comprises an increase in T-cell response as compared to a prevaccination timepoint, or
wherein the effective amount is about 2×10⁵ FFU and eliciting the immune response comprises an increase in T-cell response as compared to a prevaccination timepoint.

23. (canceled)

24. (canceled)

25. (canceled)

26. (canceled)

27. The method of claim 1,
wherein the effective amount is about 2×10⁶ FFU and eliciting the immune response comprises at least a 2.4 fold rise in IgA levels in the subject, or
wherein the effective amount is about 2×10⁵ to 2×10⁶ FFU and eliciting the immune response comprises at least a 1.3 fold rise in IgA levels in the subject, or wherein the effective amount is about 10⁵ FFU and eliciting the immune response comprises at least a 1.1 fold rise in IgA levels in the subject, or
wherein the effective amount is about 2×10⁵ FFU and eliciting the immune response comprises at least a 1.1 fold rise in IgA levels in the subject, or
wherein the effective amount is about 10⁵-10⁶ FFU and eliciting the immune response comprises at least a 1.3 fold rise in IgA levels in the subject, or
wherein the effective amount is about 2×10⁵ to 2×10⁶ FFU and eliciting the immune response comprises at least a 1.3 fold rise in IgA levels in the subject.

28. (canceled)

29. (canceled)

30. (canceled)

31. (canceled)

32. (canceled)

33. The method of claim 1, wherein the deoptimized RSV comprises the genome or anti-genome having the sequence of SEQ ID NO:1.

34. The method of claim 1, wherein the subject is under the age of 18, 13, 7, or 2.

35. (canceled)

36. (canceled)

37. (canceled)

38. The method of claim 1, wherein the subject is between 18-49 years old.

39. The method of claim 1, wherein the subject is at least 50 years old.

40. A method of eliciting an T-cell immune response in a subject, comprising:
intranasally administering a first dose of an effective amount of a composition comprising a deoptimized respiratory syncytial virus (RSV) to the subject to elicit the T-cell immune response; and
intranasally administering a second dose of the effective amount of the composition comprising the deoptimized RSV about 28-35 days after the first dose,
wherein the effective amount is about 10⁶ or about 2×10⁶ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint.

41. A method of eliciting an T-cell immune response in a subject, comprising:
intranasally administering a first dose of an effective amount of a composition comprising a deoptimized respiratory syncytial virus (RSV) to the subject to elicit the immune response;
intranasally administering a second dose of the effective amount of the composition comprising the deoptimized RSV about 28-35 days after the first dose,
wherein the effective amount is about 10⁵ or about 2×10⁵ focus forming units (FFU) of the deoptimized RSV, wherein the deoptimized RSV comprises the genome or anti-genome having the sequence of SEQ ID NO:1, and wherein eliciting the T-cell immune response comprises an increase in T-cell response as compared to a prevaccination timepoint.

42. A method of eliciting an IgA immune response, cellular immune response or both in a subject, comprising:
intranasally administering a first dose of an effective amount of a composition comprising a deoptimized respiratory syncytial virus (RSV) to the subject to elicit the immune response; and
intranasally administering a second dose of the effective amount of the composition comprising the deoptimized RSV about 28-35 days after the first dose;
wherein the effective amount is about 2×10⁵ focus forming units (FFU) of the deoptimized RSV, wherein the deoptimized RSV comprises the genome or anti-genome having the sequence of SEQ ID NO:1, and wherein an enzyme-linked immune absorbent spot (ELISpot) analysis of peripheral blood mononuclear cells (PBMCs) from the subject shows at least a 1.3 fold increase of spot forming units (SFU) between a baseline value and a highest value during a post vaccination timepoint,
OR
wherein the effective amount is about 2×10⁶ focus forming units (FFU) of the deoptimized RSV, wherein the deoptimized RSV comprises the genome or anti-genome having the sequence of SEQ ID NO:1, and wherein eliciting the immune response comprises at least a 2.4 fold rise in IgA levels in the subject.


SEQUENCE LISTING (1 sequence)

SEQ ID NO: 1 - DNA, 15111 bp, Artificial Sequence
  synthetic contruct
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacagaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcggttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagtagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaaaaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaacgtatacttaaccgatagttatttaaaaggcgtaatcagttttagcgaatgtaacgcattagggtcatatatctttaacggtccatatcttaaaaacgattatactaatctaatcagtagacagaatccgttaatcgaacatatgaatcttaagaaactgaatatcacacaatctttgatcagtaagtatcataaaggcgaaatcaaactcgaagaacctacatattttcaatcactattaatgacatataagtctatgacatctagcgaacagatcgctactactaatctgttgaagaaaattattagacgagctatagagatatctgacgttaaggtatacgctatactgaataaattggggttaaaagagaaagataagataaaatctaataacggtcaagacgaagataatagtgtaattactacaattattaaagacgatatactatccgcagtgaaggataatcaatcacatcttaaagccgataaaaatcatagtactaaacaaaaagatacaattaaaactacattgttaaagaaattgatgtgttctatgcaacatccacctagttggttaatacattggtttaacttatacactaagttgaacaatatacttacacaatatcgatcaaacgaagtgaaaaatcacggttttacattgatagataatcaaacattaagcggatttcaattcatacttaaccaatacggatgtatagtgtatcataaagaattgaaacgtataaccgttacaacatataatcaattcttaacatggaaagatataagtctatctagattgaacgtatgcttaattacatggatttcgaattgtcttaatacacttaataaatcattagggttaagatgcggatttaataacgttatacttacacaattgttcttatacggagattgtatacttaagttgttccataacgaagggttttatataataaaagaggttgagggatttataatgtcattgatactgaatattaccgaagaggatcaatttagaaaaagattctataatagtatgttaaacaatataactgacgcagctaataaagcgcagaagaatctgttatctagagtatgtcatacattgttagacaaaacagtgagcgataatattataaacggtagatggattatactgttatctaaattcttaaaattgattaagttggcaggtgacaataaccttaataacttaagcgaattgtatttcttattcagaatattcggacatcctatggttgacgaacgacaagctatggacgcagtgaagattaattgtaacgaaactaaattctatctattatctagtctatctatgcttagaggcgcattcatatatagaattataaaagggttcgttaataattataatagatggcctacacttagaaacgctatagtgttaccacttagatggttaacatattataaattgaatacatatcctagtttactcgaattaaccgaacgcgatctgatagtgttaagcggacttagattctatagagagtttagattgcctaagaaagtcgatctcgaaatgataattaacgataaggcaattagtccacctaaaaacttaatatggacaagcttccctagaaattatatgcctagtcatatacaaaattatatcgaacacgaaaaattgaaatttagcgaatccgataagtctagaagagtgttagagtattacttacgcgataataaatttaacgaatgcgatctatataattgcgtagtgaaccaatcatatcttaataatcctaatcacgtagtgagtcttacaggtaaggaaagagagttgagcgtaggtagaatgttcgctatgcaacccggtatgtttagacaagtgcaaatactcgcagaaaagatgatagccgaaaatatactgcaattctttcccgaatcattgactagatacggagatttagaattgcaaaagatactcgaattgaaagcaggtatatctaataagtctaatagatataacgataattataataattatatatctaagtgtagtattattaccgatctatctaaattcaatcaggcatttagatacgaaactagttgtatatgctcagacgtattagacgaattacacggagtgcaatctttgtttagttggttacatttaactatacctcacgttacaattatatgtacatatagacacgcaccaccatatataggcgatcatatagtcgatctgaataacgtagacgaacaatccggattgtatagatatcacatgggtggcatagagggatggtgtcaaaaattgtggactatagaggcaattagtctgttagatctaattagtcttaagggtaagttttcgattaccgcattgattaacggtgataatcaatcaattgatatatctaaaccgatacggttaatggagggacaaacacacgctcaagccgattacttactcgcacttaattcacttaaactgttatacaaagagtacgcaggtatagggcataaacttaagggtacagagacatatataagtagggatatgcaatttatgagtaagactatacaacataacggagtgtattatcccgctagtataaagaaagtgcttagagtcggaccttggattaatactatattagacgattttaaggttagtctcgaatcaatcggatcattgacacaagagttggagtatagaggcgaatctctattatgctcattgatttttagaaacgtatggttatacaatcagattgcattgcaattgaaaaatcacgcactatgtaataataagttgtacttagacatacttaaagtgttaaaacatcttaaaacattctttaatctcgataatatagataccgcattaacattgtatatgaatctacctatgttattcggagggggagatcctaatctattgtatagatcattctatagacgtacacctgatttcttaaccgaagctatagtgcatagcgtattcatactatcatattatactaatcacgatcttaaagataagttgcaggatctatctgacgatagattgaataaattcttaacatgtattataacattcgataaaaatcctaacgctgaattcgttacacttatgagagatccacaagcattaggttcagagagacaggctaaaattactagcgaaattaatagattagccgttaccgaagtgttaagtaccgcacctaataagatattctctaaatccgctcaacattatacaacaaccgaaatagatcttaacgatattatgcaaaatatcgaacctacatatcctcacggattacgcgtagtttacgaatcattaccattctataaagccgaaaagatcgttaacttaattagcggtacaaaatcaattactaatatactcgaaaagactagcgcaattgatttaaccgatatagatagagctatcgaaatgatgcgtaaaaatataacattactgatacgtatactaccattagattgtaatagggataaaagagagatactatctatggagaatctatcaattacagaattgtcaaaatacgttagggaacgatcatggtcactatctaatatcgtaggcgtaactagtcctagtattatgtatactatggatattaagtatacaactagtacaattagtagcggtataataatcgaaaaatataacgttaatagtctaacacgtggtgaaaggggacctacaaaaccttgggtcggatctagtacacaagagaagaaaactatgcccgtatataatagacaggtattgactaagaaacaacgagatcaaatagatctattagctaaactcgattgggtatacgctagtatagataataaagacgaatttatggaagagttgtcaatcggtacattagggttaacatacgaaaaagctaagaaattgttcccacaatatctatcagtgaattatctacatagattgacagtgagtagtagaccatgcgaatttcccgctagtatacccgcatatagaactactaattatcatttcgatactagtccaattaatagaatattaaccgaaaaatacggagacgaagatatagatatcgtattccaaaattgtattagtttcggattgagtcttatgtccgtagtcgaacaatttactaacgtatgtcctaataggattatactgatacctaaattgaacgaaatacatcttatgaaacctcctatttttacaggcgatgtcgatatacacaaattgaaacaggttatacaaaaacaacatatgttcttacccgataagatatcgttaacgcaatacgttgagttgttcttatcaaataaaacacttaaatcaggtagtcacgttaatagtaatctgatactcgcacataaaattagcgattactttcataatacatatatattgagtactaacttagccggacattggatactgattatacaattgatgaaagatagtaagggtatattcgaaaaagattggggtgagggatatataaccgatcatatgtttataaaccttaaggtcttctttaacgcatataaaacttatctattatgttttcataagggatacggtaaggctaaactcgaatgcgatatgaatacatccgatctattatgcgtactcgaattaattgatagtagctattggaaatctatgagtaaggtattcttagagcaaaaggtgatcaagtatatactatctcaagacgctagtttgcatagggttaagggatgtcatagttttaaattatggtttcttaaaagattgaacgtagccgaatttacagtatgtccttgggtcgttaacatagattatcatcctacacatatgaaagctatacttacatatatagatctagtgagaatgggattgattaacatagatagaatacatataaagaataaacataaatttaacgacgaattctatactagtaatctattctatataaattataatttttccgataatacacatctattaactaaacatatacgtatagctaatagcgaactcgaaaataattataataaattgtatcatcctacacccgaaacattagagaatatactcgctaatccgattaaatctaacgataagaaaacacttaacgattattgtataggtaaaaacgttgattcaattatgttaccattactatcaaataagaaattgattaaatctagcgctatgattagaactaattatagtaaacaggatctatataacttattccctatggtcgtaattgatagaattatagatcattccggtaataccgctaaatctaatcaattgtatacaactactagtcatcaaatatcattagtgcataatagtactagtctatattgtatgttaccatggcatcatattaatagattcaatttcgtttttagtagtacagggtgtaaaattagtatagagtatatacttaaagatcttaaaattaaagatcctaattgtattgcattcataggcgaaggcgcaggtaatctgttacttagaacagtagtcgaattgcatcccgatattagatatatatatagatcacttaaagattgtaacgatcatagtctaccaatcgaattccttagattgtataacggtcatataaacatagattacggcgaaaacttaacgatacccgctactgacgctactaataatatacattggtcatacttacatattaaattcgcagaacctataagtctattcgtatgcgacgcagaattatccgttacagtgaattggtctaaaattattatcgaatggtctaaacacgttagaaaatgcaaatattgttctagcgttaataagtgtatgttaatcgttaagtatcacgctcaagacgatatagattttaaattagataatataactatacttaaaacatacgtatgcttaggtagtaagcttaagggtagcgaagtatacttagtgttaacgataggtccagctaatatttttcccgtttttaacgtagtgcaaaacgctaaattgattctatctagaactaaaaattttataatgcctaagaaagctgataaagagtcaattgacgctaatataaaatcattgataccattcttatgttatcctataactaagaaagggattaataccgcactatctaaacttaaatccgtagtgagcggagatatactatcttatagtatagccggtagaaacgaagtttttagtaataaattgattaatcataaacatatgaatatacttaaatggtttaatcacgtacttaattttagatcaaccgaattgaattataatcatctatatatggtcgaatctacatatccatacttatccgaactgttaaactcattgactactaacgaattgaagaaattgattaaaattacaggtagtctgttatacaattttcataacgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt